(19) United States
(12) Patent Application Publication
(10) Pub. No.: US 2022/0387366 A1
(43) Pub. Date: Dec. 8, 2022

(54) Compositions for Regulating Homeostasis of Cortisol and Improving Sleep Quality and Methods of Use and Production Thereof

(71) Applicant: Unigen, Inc., Tacoma, WA (US)

(72) Inventors: Mesfin Yimam, Tacoma, WA (US); Teresa Horm, Tacoma, WA (US); Qi Jia, Tacoma, WA (US); Lidia Alfaro Brownell, Tacoma, WA (US); Ping Jiao, Tacoma, WA (US)

(73) Assignee: Unigen, Inc., Tacoma, WA (US)

(21) Appl. No.: 17/831,862

(22) Filed: Jun. 3, 2022

Related U.S. Application Data
(60) Provisional application No. 63/196,398, filed on Jun. 3, 2021.
(60) Provisional application No. 63/293,856, filed on Dec. 27, 2021.

(51) Int. Cl. A61K 31/198; A61K 31/165; A61K 36/899; A61P 25/20

(52) U.S. Cl. CPC A61K 31/198; A61K 31/165; A61K 36/899; A61P 25/20

(57) ABSTRACT

Compositions used and methods are disclosed for regulation of homeostasis of host cortisol and improving sleep quality including a composition derived from enriched for one or more phenylpropanoid acids and benzoxazinoids or extracts that are enriched for one or more phenylpropanoid acids and benzoxazinoids. Compositions of enriched for one or more phenylpropanoid acids and benzoxazinoids maintain homeostasis of host stress hormone, cortisol, selectively binds to MT2 over MT1 receptor, improves sleep quality by enhancing the deep sleep stage of sleep, increases total sleep time and deep sleep time, improves overall mental well-being measured by the Pittsburgh Sleep Quality Index (PSQI) and Profile of Mood States (POMS), provides positive mood support and enhances emotional well-being; maintains homeostasis of biomarkers—serotonin, melatonin, GABA in formulation in a mammal disclosed that includes administering an effective amount of a composition from 0.01 mg/kg to 1000 mg/kg body weight of the mammal.

This United States Utility Application claims priority to U.S. Provisional Application Ser. No. 63/196,398 filed on Jun. 3, 2021 and entitled “Compositions and Methods for Regulating Homeostasis of Cortisol and Improving Sleep Quality”, and U.S. Provisional Application Ser. No. 63/293,856 filed on Dec. 27, 2021 and entitled “A 6-MBOA Standardized Dietary Supplement for Improved Sleep Quality and Mood States”, both of which are commonly owned and incorporated by reference in their entirety herein.

### FIELD OF THE SUBJECT MATTER

The field of the subject matter is compositions and methods of use and production thereof for regulating homeostasis of cortisol and improving sleep quality.

### BACKGROUND

Sleep disturbance and deprivation is one of the primary psychiatric complaints associated with cognitive impairment, daytime sleepiness, occupational hazard, loss of productivity, and traffic accidents (Durmer and Dinges, 2005). While behavioral techniques, such as improving sleep hygiene are generally the first line of intervention, numerous types of medications are frequently used as adjuncts. Prescribing sedating antidepressants such as the tricyclic antidepressants (e.g., amitriptyline and doxepin), the tetracyclic antidepressant (e.g., mirtazapine) and the serotonin antagonist and reuptake inhibitor (e.g., trazodone) are becoming the mainstream practice for patients with insomnia. Besides their daytime residual effect, drug dependency and long-term adverse consequences, the use of antidepressants in nondepressed patients raises ethical questions and remains controversial. As a result, natural sleep aids are widely used as alternatives to prescription drugs to improve the sleep quality and to avoid side-effects, including impaired cognitive function, tolerance, and dependence.

Sleep disturbance is highly prevalent and believed to affect both physical and mental health where the collective knowledge suggests that altered functioning of the hypothalamic-pituitary-adrenal (HPA) axis might underlie this association. The hypothalamic-pituitary-adrenal (HPA) axis is an adaptive system with a key role in maintaining physiological homeostasis. A dysfunction or disturbance in this vital system will have some consequential outcomes in the natural homeostasis process. Sleep is among several physiologic functions regulated by HPA axis. In this axis, corticotropin-releasing hormone (CRH) is secreted by the paraventricular nucleus (PVN) located in the hypothalamic region of the brain and acts on CRH receptors in the anterior pituitary gland to cause the release of adrenocorticotropic hormone (ACTH). ACTH acts on the adrenal cortex, which produces and releases cortisol. Cortisol then will deliver the feedback to the body for the normal physiologic sleep and awake order through HPA axis. While sleep initiation occurs when HPA axis activity is down, sleep deprivation and/or nighttime awakening is association with HPA activation. Impaired HPA axis in association with pulsatile cortisol level is one of the primary causes of sleep disturbances and fragmentation. Increased activation of the hypothalamic-pituitary-adrenal (HPA) axis expressed as elevated plasma cortisol levels was shown in physiological ageing. Hence, through stabilization of HPA axis dysfunction, reducing the cortisol level could be considered as an effective approach to address sleep disturbances.

Sleep has a modulatory effect on many segments of the neuroendocrine system, where many hormones secreted as a result of activation of this system affect sleep and vice versa (van Dalfsen et al., 2018). It has been well documented that sleep displays a close and reciprocal relationship with the functioning of the HPA axis (Balbo et al., 2010). In general, activation of the hypothalamic-pituitary-adrenal axis is known to lead to arousal and sleeplessness. Cortisol secretion through the effect of HPA axis is amongst the hormones that affect the human daily cycle including sleep. Cortisol is one of the major glucocorticoid hormones secreted by the adrenal cortex where its levels rise before dawn, rapidly increase after awakening, and decrease over the course of the day with a nadir early in the sleep period. Although the levels of cortisol show a high degree of variability between subjects, a given individual tends to have a consistent rhythm. As a result, measuring the plasma cortisol level is a good predictor of sleep quality and its effect on HPA axis. For instance, in a clinical setting, when 33 healthy young men were subjected to a partial and total sleep deprivation, there were statistically significant increase in plasma cortisol levels on the next day for both the scenarios suggesting that even partial acute sleep loss delays the recovery of the HPA from early morning circadian stimulation. Based on these findings, the authors concluded that the increase in plasma cortisol level and delayed HPA response could likely to involve an alteration in negative glucocorticoid feedback regulation (Leproult et al., 1997). This is a clear indication that increased sleep fragmentation evidenced by decreased slow wave sleep and sleep loss could contribute to elevated cortisol level. Thus, blunting the cortisol level and hence stabilizing the HPA axis feedback for a rapid recovery is a key step for managing sleep disturbances for a better and improved sleep quality and efficiency.

The human sleep and wake cycle is under the dual control of the circadian rhythmicity and sleep homeostatic mechanisms. These two courses are known to work independently, though together, they determine most aspects of sleep and related variables. While slow wave deep sleep is primarily controlled by the homeostatic process, sleep timing is monitored by circadian rhythmicity (Deboer, 2018). Changes in the cortisol level is an indication of alteration of the homeostatic process and hence impairment of the negative feedback control of the HPA axis which could promote fragmentation and poor quality of sleep. Elevated level of cortisol is associated with sleep fragmentation and wakefulness.

In addition, following a functional link between sleep and cortisol secretion as the mode of action of antidepressants in insomnia, the effects of the tricyclic antidepressant doxepin were investigated on nocturnal sleep and plasma cortisol concentration in middle aged patients with chronic primary insomnia (Rodenbeck et al., 2003). It was found that, subjects who received oral doxepin for 3 weeks showed significantly improved sleep and reduced mean cortisol levels. According to the authors, the results implicate that the sleep-improving effects of doxepin are mediated at least in part by a normalization of hypothalamic-pituitary-adrenal axis functions. Thought the study was conducted on subjects with insomnia, these findings seem to mirror clinical results disclosed herein with improved sleep quality observations found as a result of contemplated compositions derived from enriched for one or more phenylpropanoid acids and benzoxazinoids supplementation in healthy subjects. A similar clinical study was also carried out on insomniac but nondepressed subjects and determined the association of chronic insomnia with increased plasma levels of ACTH and cortisol where activation of the hypothalamic-pituitary-adrenal axis was considered the main pathway (Vgontzas et al., 2001). Here again, elevation in cortisol may be a primary cause of the sleep disturbance and may be a marker for increased CRH activity.

Normal human sleep involves two states such as rapid eye movement (REM) and non-REM (NREM) sleep that alternate regularly across a sleep episode. Physiologically, normal sleep is characterized by cycles of light sleep (stages 1 and 2), deeper slow-wave sleep (stages 3 and 4), and rapid eye movement (REM) sleep throughout the night (Carskadon M A, Dement, 2000). In general, in adults, about 75-80% of total time spent in sleep is spent in NREM sleep while the remaining 20-25% happens in REM sleep. Usually, we spend considerable amount of time in slow-wave deep sleep (SWS) each night which is critically important for cerebral restoration and recovery, the maintenance and consolidation of memory, and metabolic regulation (Stickgold, 2005; Tasali et al., 2008). These facts suggest that for a better quality of sleep and efficiency, it is preferable to influence the NREM sleep in particular the deep sleep. Each stage of sleep has a defined electroencephalogram (EEG) frequency and waveform. While increased EEG frequency is associated with wakefulness, decreased EEG frequency is associated with increased depth of sleep. As such, factors that increase EEG frequency will tend to negatively impact sleep, yielding lighter sleep and wakefulness. Increased corticotropin-releasing hormone (CRH) and hence increased cortisol level appears to be one common factor to increase sleep EEG and thereby increase wakefulness through HPA axis dysregulation. It impairs sleep quality and efficiency. For instance, intravenous administration of exogenous CRH to healthy subjects was found to cause decreased slow wave deep sleep and increase light sleep and awakenings (Holsboer et al., 1988).

Poor sleep quality is one of the most common health complaints of older people. Shortened nocturnal sleep duration, increased frequency of daytime naps, increased number of nocturnal awakenings and time spent awake during the night, and decreased amounts of deep slow wave sleep are the most frequent changes associated with normal aging (Li et al., 2018). In contrast, among the different stages of sleep, the REM sleep seem to be relatively better conserved. For example, using data combined from a series of clinical studies conducted between 1985 and 1999, Cauter et al., reported the chronology of age-related changes in sleep quality in healthy men and associated alterations in cortisol level. In this analysis, slow wave deep sleep decreased sharply from early adulthood to midlife, whereas wake time increased and REM sleep declined by about 30 and 10 min, respectively, per decade from midlife to late life. Analysis of the 24-hour mean cortisol level per decade for midlife to late midlife men were markedly associated with decrease in slow wave deep sleep indicating the strong correlation of poor quality of sleep and increased level of plasma cortisol (Van Cauter et al., 2000).

The concept of daytime cortisol level and its correlation to sleep disturbance were also assessed in healthy older subjects who frequently report declined sleep quality (Morgan et al., 2017). A total of 672 older adults with age group of 67-90 were included in this study. Regression analyses were conducted for a daytime cortisol level and sleep characteristics (fragmentation, wake after sleep onset and duration) which were derived from wrist actigraphy. It was found that both higher fragmentation score and longer wake after sleep onset were significantly associated with higher daytime cortisol; however, sleep duration was not. In a similar study, compared with healthy young subjects (average of 21 years old), healthy older adults (average of 71 years old) were found slept poorly at night as indicated by increased wake time and percentage stage 1 sleep and decreased percentage of slow wave sleep. There was also a stronger association of cortisol with total wake time in the older individuals (Vgontzas et al., 2003). Similarly, interaction between altered HPA-axis functions and disturbed sleep was underlined by studies indicating a positive correlation between total wake time and the 24-h urinary cortisol secretion (Vgontzas et al., 1998) and increased evening/nocturnal plasma cortisol levels with the number of nocturnal awakenings (Rodenbeck et al., 2002). These reports support the hypothesis that poor sleep quality is closely linked to the cortisol level where reduced cortisol level is a fundamental step for maintaining sleep homeostasis and hence improved sleep quality. In fact, our clinical study result showed that subjects who were supplemented with a composition derived from enriched for one or more phenylpropanoid acids and benzoxazinoids to have significantly reduced cortisol level and a better sleep quality and presumed normalized HPA activity as reflected by the longer slow wave deep sleep time. These observations cannot be simply explained by the association that actives in a composition derived from enriched for one or more phenylpropanoid acids and benzoxazinoids would mimic melatonin activity to produce its effects whereas it could be indeed by maintaining a homeostatic function of HPA axis to yield an improved sleep quality and efficiency. Effect on sleep time which mainly regulated by the circadian rhythmicity was moderate in our study. Substantiating the current work, which is disclosed herein, it has been reported that in contrast to the quality of sleep, normal variations in sleep quantity (i.e., sleep duration) was not found to influence cortisol responsiveness (Bassett et al., 2015).

### SUMMARY OF THE SUBJECT MATTER

Compositions are disclosed herein comprising an extract, wherein the extract is enriched for one or more phenylpropanoid acids and one or more benzoxazinoids for establishment and regulation of homeostasis of host stress hormone, cortisol, and improvement of sleep quality.

In other embodiments, contemplated compositions comprise an extract from corn leaf or corn shoots, wherein the extract is enriched for one or more benzoxazinoids comprising both aglycones of benzoxazoles, benzoxazinonoids, benzoxazolinonoids, or glycosides of benzoxazole, benzoxazinonoids, and benzoxazolinonoids.

In some embodiments, contemplated compositions comprise a corn shoot or corn leaf extract, wherein the extract is enriched for one or more phenylpropanoid acids and one or more benzoxazinoids.

In yet other embodiments, contemplated compositions are enriched for one or more phenylpropanoid acids and one or more benzoxazinoids for establishment and regulation of homeostasis of host stress hormone, cortisol, and improvement of sleep quality.

In other embodiments, contemplated compositions comprise at least one phenylpropanoid acid and at least one benzoxazinoid.

In yet other embodiments, contemplated compositions comprise at least one phenylpropanoid acid and at least one benzoxazinoid, wherein the at least one phenylpropanoid acid and the at least one benzoxazinoid are enriched for in the composition.

### BRIEF DESCRIPTION OF THE FIGURES

FIG. 1 shows the supplementation of a composition derived from enriched for one or more phenylpropanoid acids and benzoxazinoids at 250 mg/day showed statistically significant increase in the deep sleep time compared to placebo on weeks 2 (p=0.0219) and 3 (p=0.0147) with a positive trend on week 4 (p=0.0621). Supplementation of a composition derived from enriched for one or more phenylpropanoid acids and benzoxazinoids at 500 mg/day showed statistically significant increase in the deep sleep time compared to placebo on week 3 (p=0.0131) with positive trends on week 2 (p=0.057) and week 4 (p=0.0554). The increase in deep sleep time among all the groups was found statistically significant (p=0.0318).

FIG. 2 shows the total sleep time and compared to the placebo group, statistically significant increase in the total sleep time was observed for participants supplemented with 500 mg/day a composition derived from enriched for one or more phenylpropanoid acids and benzoxazinoids with p=0.050 across the weeks.

FIG. 3 shows the supplementation of a composition derived from enriched for one or more phenylpropanoid acids and benzoxazinoids at 250 mg/day showed statistically significant improvement in quality of sleep as measured in PSQI compared to placebo on weeks 3 (p=0.0325) with positive trends on 2 (p=0.0690) and week 4 (p=0.0584). Supplementation of a composition derived from enriched for one or more phenylpropanoid acids and benzoxazinoids at 500 mg/day showed statistically significant improvement in quality of sleep as measured in PSQI compared to placebo on week 3 (p=0.0022) with a positive trend on week 4 (p=0.0756). The improvement in quality of sleep as measured by PSQI among the groups was found statistically significant with P=0.0273.

FIG. 4 shows the supplementation of a composition derived from enriched for one or more phenylpropanoid acids and benzoxazinoids at 500 mg/day showed statistically significant decrease in the salivary cortisol level compared to placebo on week 4 (p=0.0229) with positive trends on week 2 (p=0.0757) and week 3 (p=0.0604). The decrease in salivary cortisol level among all the groups was found statistically significant (p=0.0289).

FIG. 5 shows that compared to placebo, participants supplemented with a composition derived from enriched for one or more phenylpropanoid acids and benzoxazinoids at 250 mg/day showed statistically significant increase in the overall wellbeing across the weeks with P=0.0020, 0.0117, 0.0006 and 0.0318 for weeks 1, 2, 3 and 4, respectively. Compared to placebo, participants supplemented with a composition derived from enriched for one or more phenylpropanoid acids and benzoxazinoids at 500 mg/day showed statistically significant increase in the overall wellbeing on weeks 1 and 3 with P=0.0240, and 0.0028, respectively. The improvement in the overall wellbeing among groups was statistically significant (p=0.0003).

### DETAILED DESCRIPTION

Compositions used and methods are disclosed for regulation of homeostasis of host cortisol and improving sleep quality including a composition derived from enriched for one or more phenylpropanoid acids and benzoxazinoids. The source of enriched for one or more phenylpropanoid acids and benzoxazinoids include but are not limited to Zea mays, Oryza species, Secale cereale, Acanthus species, Avena species, Coix lachryma-jobi, Hordeum vulgare, Triticum species, Sorghum bicolor, Lobelia chinensis, Leymus chinensis, Aphelandra spp, Scoparia dulcis, Capparis sikkimensis sp or a combination thereof.

Compositions are disclosed herein comprising an extract, wherein the extract is enriched for one or more phenylpropanoid acids and one or more benzoxazinoids for establishment and regulation of homeostasis of host stress hormone, cortisol, and improvement of sleep quality.

In other embodiments, contemplated compositions comprise an extract from corn leaf or corn shoots, wherein the extract is enriched for one or more benzoxazinoids comprising both aglycones of benzoxazoles, benzoxazinonoids, benzoxazolinonoids, or glycosides of benzoxazole, benzoxazinonoids, and benzoxazolinonoids.

In some embodiments, contemplated compositions comprise a corn shoot or corn leaf extract, wherein the extract is enriched for one or more phenylpropanoid acids and one or more benzoxazinoids.

In yet other embodiments, contemplated compositions are enriched for one or more phenylpropanoid acids and one or more benzoxazinoids for establishment and regulation of homeostasis of host stress hormone, cortisol, and improvement of sleep quality.

In other embodiments, contemplated compositions comprise at least one phenylpropanoid acid and at least one benzoxazinoid.

In yet other embodiments, contemplated compositions comprise at least one phenylpropanoid acid and at least one benzoxazinoid, wherein the at least one phenylpropanoid acid and the at least one benzoxazinoid are enriched for in the composition.

Contemplated compositions of enriched for one or more phenylpropanoid acids and benzoxazinoids maintain homeostasis of host stress hormone, cortisol, selectively binds to MT2 over MT1 receptor, improves sleep quality by enhancing the deep sleep stage of sleep, increases total sleep time and deep sleep time, improves overall mental well-being measured by the Pittsburgh Sleep Quality Index (PSQI) and Profile of Mood States (POMS), provides positive mood support and enhances emotional well-being; maintains homeostasis of biomarkers—serotonin, melatonin, GABA in formulation in a mammal disclosed that includes administering an effective amount of a composition from 0.01 mg/kg to 1000 mg/kg body weight of the mammal.

As used herein, “enriched for” refers to a plant extract or other preparation having at least a two-fold up to about a 1000-fold increase of one or more active compounds as compared to the amount of one or more active compounds found in the weight of the plant material or other source before extraction or other preparation. In certain embodiments, the weight of the plant material or other source before extraction or other preparation may be dry weight, wet weight, or a combination thereof. As used throughout this disclosure, “enriched for” doesn't mean pure concentration of the plant extract or other preparation (increasing all of the constituents by the same degree) but means amplifying or increasing one or more active ingredients as compared to the other ingredients, some of which may also be active. The act of preparing a compound or composition that is enriched for also means that the enriched for composition or compound is not found in nature—it is prepared, developed, or otherwise treated to be enriched for.

As mentioned, normal human sleep is regulated by a sleep/wake cycle homeostatic and a circadian process. These two mechanisms are known to work independently, though both influence sleep and sleep related variables. Normal human sleep stages include a non-REM (NREM) sleep and rapid eye movement (REM) sleep stages that alternate regularly across a sleep episode overnight. Among the non-REM sleep stages, the slow wave deep sleep N3) is primarily controlled by the homeostatic process through hypothalamic-pituitary-adrenal (HPA) axis feedback mechanisms, while sleep quantity is monitored by circadian rhythmicity. Cortisol, one of the major glucocorticoid hormones secreted by the adrenal cortex, is amongst the hormones that affect the human daily cycle including sleep. Increased plasma and/or salivary cortisol level is associated with impaired HPA feedback regulation that will lead to, decreased slow wave sleep, premature nocturnal awakening, sleep fragmentation and poor sleep quality culminating to a stress which the body responds by activating the HPA axis. The increased HPA activity will in turn lead to increased cortisol secretion which is a crucial factor in inducing and maintaining sleep disturbances creating a vicious cycle.

Contemplated embodiments utilize the immature corn leaf extract for regulating homeostasis of cortisol and improvement of sleep quality by breaking the vicious cycle as demonstrated in human clinical trial conducted in healthy subjects supplemented with the novel composition. Subjects who received a composition derived from enriched for one or more phenylpropanoid acids and benzoxazinoids showed statistically significant dose correlated reduction in salivary cortisol level. These subjects also experienced statistically significant increase in the deep sleep stages of sleep as early as two weeks after oral supplements. This decrease in salivary cortisol and increase in deep sleep correlation indicates the mechanisms by which a composition derived from enriched for one or more phenylpropanoid acids and benzoxazinoids produced improved sleep quality and efficiency through modulation of the homeostatic pathway of the HPA axis negative feedback regulations and reduction of cortisol.

The novelty of a composition derived from enriched for one or more phenylpropanoid acids and benzoxazinoids also arise from the fact that there have never been reports of a 4-fold higher affinity to MT2 melatonin receptor over MT1 melatonin receptor leading to a statistically significant increase in the slow wave sleep (deep sleep) stage of sleep demonstrated in human clinical studies. The use of current embodiments shows that the presence of enriched for one or more phenylpropanoid acids and benzoxazinoids other than 6-MBOA binding to melatonin receptors with observed selectivity of MT2 receptor leads to an enhanced clinically meaningful and significant increase in deep sleep time.

Supplementation or use of the contemplated composition enriched for one or more phenylpropanoid acids and benzoxazinoids (referred to in this work as UP165) improved sleep quality of clinical study participants by increasing the deep sleep stage of sleep approximately by half an hour. A 7-fold increase in deep sleep time was observed for the participants who were supplemented with the enriched for one or more phenylpropanoid acids and benzoxazinoids, UP165 at 250 mg/day compared to the placebo group at week 4. The total deep sleep times at the start of the study period for the 250 mg/day, 500 mg/day and placebo were 64, 68 and 58 minutes, respectively indicating neither the supplement nor the placebo group were to the level considered good-quality sleep per night. Following the 4-week supplementation, the deep sleep times were found to be increased to 92, 94 and 62 minutes for the 250 mg/day, 500 mg/day of the disclosed composition enriched for one or more phenylpropanoid acids and benzoxazinoids and placebo group, respectively. It is believed that to have good-quality sleep, one has to have a minimum of 90 minutes total deep sleep time per night (Vgontzas et al., 2003; Wheatley, 2005; Yaneva et al., 2004). These data clearly show that, supplementation of UP165 composition enriched for one or more phenylpropanoid acids and benzoxazinoids helped the participants to achieve a good quality of sleep per night by increasing the deep sleep stage of sleep.

As demonstrated by the clinical trial results disclosed in the current contemplated embodiments, subjects who received the composition derived from enriched for one or more phenylpropanoid acids and benzoxazinoids have showed statistically significant reduction in cortisol level that led to clinically meaningful increase in slow wave deep sleep time which is the reflection of improved sleep quality. In fact, association of these two factors (cortisol level and sleep quality) were observed in our clinical trial which produced reduced salivary cortisol level and improved slow wave deep sleep and hence better sleep quality. In the clinical trial disclosed in current contemplated embodiments, subjects who were supplemented with a composition (UP165) derived from corn leaf or shoot extracts that are enriched for one or more phenylpropanoid acids and benzoxazinoids showed significant inverse correlation between salivary cortisol level and deep sleep suggesting improved sleep quality and efficiency.

In the current contemplated embodiments, the regular day long decline in cortisol concentrations that follows the early morning circadian elevation appeared to occur at a slower rate and/or remained elevated for the placebo group. In contrast, subjects who received a dose of UP165 composition derived from enriched for one or more phenylpropanoid acids and benzoxazinoids experienced a faster recovery and assumed a relatively normal homeostatic function as documented by reduced level of salivary cortisol. These findings were in accordance with slow wave deep sleep pattern observations which were significantly longer in duration compared to the placebo group. The overall data interpretation in the current contemplated embodiments highlights the importance of reducing cortisol level for an improved sleep quality and efficiency independent of circadian rhythmicity.

The sleep aid effects of different doses of corn leaf or corn shoots extracts enriched for one or more phenylpropanoid acids and benzoxazinoids were assessed in mice with or without pentobarbital treatment for sleep time and latency. Data depicted here demonstrated that contemplated compositions potentiated pentobarbital-induced sleep behaviors in mice. The duration required to fall asleep was also reduced as a result of treatment in mice at subhypnotic state with contemplated compositions. Contemplated enriched for extracts disclosed herein potentiated pentobarbital-induced sleep at all the dosages tested (250-1000 mg/kg) that was designed based on calculation of 6-MBOA at 0.2% concentration in these contemplated extracts with equivalent melatonin equivalent amount of 6-MBOA between 0.5-2 mg/kg.

However, a single oral administration of a contemplated composition at a dose level as high as 1000 mg/kg did not cause drowsiness or induce an immediate sleep. Nevertheless, this preclinical study would extrapolate an extremely high human daily dosage at least 1,250 mg/day based on the minimum efficacious dose of 250 mg/kg in the animal study converted into human equivalent daily dose and could not explicitly predict and teach away the significance of usage in sleep aids for UP165 composition based on the content of 6-MBOA on sleep quality and efficiency.

As illustrated in the human clinical trial data of current contemplated embodiments, subjects who were supplemented with UP165 enriched for one or more phenylpropanoid acids and benzoxazinoids did experience the increases in total sleep time, significantly increased deep sleep time, reduced salivary cortisol, and improved mental wellbeing at a dosage as low as 200 mg per day that is 6.25 folds less the extrapolated human efficacious dosage based on the in vivo sleep study use 6-MBOA as active component.

This discrepancy could be also indicated by the fact that the pentobarbital induced sleep model may not be the right model to predict or extrapolate the usage of a composition in human sleep quality or the mechanism by which contemplated compositions imposed their effects could be different from the melatonin which was used as appositive control. Unlike the preclinical in vivo study, which was pentobarbital induced, the clinical study followed the natural behavioral sleep pattern of subjects without exogenous sleep drug induction. As the data shows for the current contemplated embodiments, the improved sleep quality and efficiency observed in the clinical trial with reduction of the level of cortisol from UP165 composition derived from enriched for one or more phenylpropanoid acids and benzoxazinoids oral administration were unexpected from the in vivo pentobarbital induced sleep study of animals.

The physiological effects of melatonin in the brain result from the activation of high-affinity, G protein-coupled receptors, referred to as MT1 and MT2. MT1 and MT2 receptors have specific roles in the modulation of sleep. The activation of the MT1 receptors are mainly implicated in the regulation of rapid eye movement (REM) sleep, whereas the MT2 receptors selectively increase non-REM (NREM) sleep. As a result, selective ligands could have therapeutic potential for sleep. While the MT2 agonists or partial agonists might be indicated for NREM-related sleep, the MT1 agonists or partial agonists might be designated for REM-related sleep disturbances (Gobbi, Comai, 2019). In the first of the receptor binding assays conducted specifically in the current contemplated embodiments for MT1 receptor, contrary to what has been reported on the literature and predicted in the issued US patents (Rosenfeld 2009 and Shelby 2020), interestingly, we found inhibition of 2-lodomelatonin binding to MT1 receptor more than the predicted affinity by contemplated compositions derived from corn leaf or corn shoots extracts enriched for one or more phenylpropanoid acids and benzoxazinoids. Concentrations for the receptor binding assay for the composition derived from enriched for one or more phenylpropanoid acids and benzoxazinoids and 6-MBOA were selected based on the amount of the standardized constituent (0.2% 6-MBOA) present in the composition derived from enriched for one or more phenylpropanoid acids and benzoxazinoids.

A composition derived from corn leaf or corn shoots extracts enriched for one or more phenylpropanoid acids and benzoxazinoids inhibited 2-lodomelatonin binding to MT1 at 50 and 100 μg/mL, two concentrations that corresponded to 0.815 μM and 1.63 μM 6-MBOA. At these concentrations, 6-MBOA surprisingly failed to inhibit the binding of 2-lodomelatonin to MT1 at any concentration. This indicated that there were components of the composition derived from enriched for one or more phenylpropanoid acids and benzoxazinoids other than 6-MBOA that competitively bound to the MT1 receptor. As such, it can be inferred that there are unexpected active compounds other than 6-MBOA in a contemplated composition derived from corn leaf or corn shoots extracts enriched for one or more phenylpropanoid acids and benzoxazinoids work in concert to produce clinically meaningful sleep quality. Interestingly, the composition derived from enriched for one or more phenylpropanoid acids and benzoxazinoids also showed a 4-fold increase in MT2 receptor binding affinity than MT1.

A composition derived from corn leaf or corn shoots extracts enriched for one or more phenylpropanoid acids and benzoxazinoids had a dose-responsive curve with an IC50 of 229 μg/mL and an inhibition constant (Ki) of 119 μg/mL for MT1 receptor and an IC50 of 56.6 μg/mL with inhibition constant Ki: 28.3 μg/mL for MT2. As seen in the clinical data, compared to the Placebo group, UP165-supplemented participants experienced an extended period of the deep sleep stage of sleep, which, in turn, assisted the participants to be better prepared for enhanced next-day performance and general well-being. The increase in the deep sleep time observed in the UP165-supplemented group has certainly a direct reflection of the 4-fold higher affinity of UP165 to the MT2 receptor, which is known to promote deep sleep when activated.

As shown in Example 6, bioassay guided isolation and purification of compounds in the active fractions from UP165 composition led to purified individual phenylpropanoid acids and benzoxazinoids with much reduced potency for melatonin receptor binding activity compared to the fraction only contain both phenylpropanoid acids and benzoxazinoids. Therefore, the co-existence of both phenylpropanoid acids and benzoxazinoids are essential for the melatonin receptor binding activity. These two types of molecules phenylpropanoid acids and benzoxazinoids could work as ‘prodrug” mechanism to act together on binding of the melatonin receptors after administration.

Data depicted in these contemplated embodiments (double-blind, placebo-controlled clinical trial) have shown that supplementation of participants with contemplated compositions, enriched for one or more phenylpropanoid acids and benzoxazinoids, produced a statistically significant improvement in sleep quality compared to the placebo group. By the end of the 4-week supplementation period, participants in the UP165 group benefited significantly more than those in the placebo group, both in total sleep time, sleep quality and overall well-being.

As a considerable amount of time is spent in the non-REM stage of sleep, wherein slow-wave deep sleep (SWS) stage is present, products with a direct impact on this stage will have clinically meaningful outcomes for cerebral restoration and recovery, maintenance and consolidation of memory, cellular rejuvenation, immune strengthening, and vital metabolic regulation. Failure to attain normal physiological recuperation as a result of fragmented and poor-quality sleep causes severe consequences on the overall mood state and well-being of participants. The improved sleep quality findings such as the deep sleep and total sleep time from the sleep tracker were also verified by the PSQI questionnaire with participants showing a 10-fold increase in quality and efficiency of sleep improvement as a result of UP165 composition enriched for one or more phenylpropanoid acids and benzoxazinoids, compared to that of the participants who were supplemented with the placebo. Again, substantiating the objective measures from the individual sleep tracker, when participants were asked about their mental health status using the Profile of Mood States questionnaire, they provided statistically significant improvements in mood state and well-being compared to their baseline (37-58% improvements at 250 mg/day and 36-42% improvement at 500 mg/day), whereas the improvements for the placebo group were very minimal (9-15% improvement) and statistically non-significant.

The disclosed composition enriched for one or more phenylpropanoid acids and benzoxazinoids is enriched for one or more benzoxazinoids, in which 6-MBOA can be utilized as a quality marker as contemplated herein. Contemplated benzoxazolinonoids are extracted from corn shoot or immature corn leaf with any suitable solvent, including water, methanol, ethanol, acetone, alcohol, a water-mixed solvent or a combination thereof or with supercritical fluid. In contemplated embodiments, the corn shoot or immature corn leaf extract comprises about 0.01% to about 99.9% benzoxazinoids.

Contemplated benzoxazinoids isolated from corn shoots or immature corn leaves are glycosides of the following benzoxazoles including but not limited to 6-methoxy-2-benzoxazolol (MBOA); 2-benzoxazolol (BOA); 4-methylbenzoxazole; 2,4-dimethylbenzoxazole; 2,6-dimethylbenzoxazole; 2,6-benzoxazolediol; 2,4-benzoxazolediol; 4-acetyl-2(3H)-benzoxazolone; 6-methoxy-N-methyl-2(3H)-benzoxazolone; 3-hydroxy-6-methoxy-2-benzoxazolin-2(3H)-one; 2-hydroxy-6,7-dimethoxybenzoxazole; 5,6-dimethoxy-2-benzoxazolinone; 3,6-dimethoxybenzoxazolin-2(3H)-one; 5-chloro-6-methoxy-2-benzoxazolinone; Trehalamine or a combination thereof.

Corn shoot or immature corn leaf extracts disclosed in current contemplated embodiments are enriched for one or more benzoxazinoid glycosides, including HMBOA-Glc, as contemplated herein. Contemplated benzoxazinonoids isolated from corn shoot or immature corn leaf extract are extracted with any suitable solvent, including water, methanol, ethanol, acetone, alcohol, a water-mixed solvent or a combination thereof or with supercritical fluid. In contemplated embodiments, the corn shoot or immature corn leaf extract comprises about 0.01% to about 99.9% benzoxazinoid glycosides, Contemplated benzoxazinoid glycosides, isolated from corn extract are glycosides of the following one or combination of more benzoxazinoid compounds including but not limited to 7-methoxy-2,7-dihydroxy-2h-1,4-benzoxazin-3(4H)-one; (R)-form 2-O-β-d-glucopyranoside (HMBOA-Glc), 2-hydroxy-2H-1,4-benzoxazin-3(4H)-one; (R)-form, 2,7-dihydroxy-2H-1,4-benzoxazin-3(4H)-one; (R)-form; N-hydroxy-2-hydroxy-2H-1,4-benzoxazin-3(4H)-one; (R)-form, 7-methoxy-2,7-dihydroxy-2H-1,4-benzoxazin-3(4H)-one; (R)-form, N-hydroxy-7-methoxy-2,7-dihydroxy-2H-1,4-benzoxazin-3(4H)-one; (R)-form, cappamensin A, N-methoxy-7-methoxy-2,7-dihydroxy-2H-1,4-benzoxazin-3(4H)-one; (R)-form, monocillinol A, monocillinol B, N-hydroxy-6,7-dimethoxy-2,6,7-trihydroxy-2H-1,4-benzoxazin-3(4H)-one; (R)-form, N-hydroxy-7,8-dimethoxy-2,7,8-trihydroxy-2H-1,4-benzoxazin-3(4H)-one; (R)-form, blepharin (HBOA-Glc), N-hydroxy-2-hydroxy-2H-1,4-benzoxazin-3(4H)-one; (S)-form-2-O-β-d-glucopyranoside, 2,7-dihydroxy-2H-1,4-benzoxazin-3(4H)-one; (R)-form, 2-O-β-d-glucopyranoside, 2,5-dihydroxy-2H-1,4-benzoxazin-3(4H)-one; (R)-form, 2-O-β-d-glucopyranoside, 6-hydroxyblepharin, 7-me ether; 2,7-dihydroxy-2H-1,4-benzoxazin-3(4H)-one; (R)-form, 2-O-β-galactopyranoside, 7-chloro-N-hydroxy-7-methoxy-2-hydroxy-2H-1,4-benzoxazin-3(4H)-one; (s)-form, glucopyranoside, 2,7-dihydroxy-2H-1,4-benzoxazin-3(4H)-one; (R)-form, glucopyranoside, 7-chloro-N-hydroxy-2-hydroxy-2H-1,4-benzoxazin-3(4H)-one; (S)-form, 2-O-β-d-glucopyranoside, benzoxacystole, 7,8-dimethoxy-2,7-dihydroxy-2H-1,4-benzoxazin-3(4H)-one; (R)-form, 2-O-β-d-glucopyranoside, N-methoxy-7-methoxy-2,7-dihydroxy-2H-1,4-benzoxazin-3(4H)-one; (R)-form, 2-O-β-d-glucopyranoside, N-hydroxy-7,8-dimethoxy-2,7,8-trihydroxy-2H-1,4-benzoxazin-3(4H)-one; (R)-form, 2-O-β-d-glucopyranoside or a combination thereof.

Corn shoot or immature corn leaf extract is enriched for one or more phenolic acids, particularly phenylpropanoid acids, including one or combination of more but not limited to cinnamic acid, coumaric acid, ferulic acid, phloretic acid, as contemplated herein. Contemplated phenylpropanoid acids isolated from corn shoot or immature corn leaf extract are extracted with any suitable solvent, including water, methanol, ethanol, acetone, alcohol, a water-mixed solvent or a combination thereof or with supercritical fluid. In contemplated embodiments, the corn shoot or immature corn leaf extract comprises about 0.01% to about 99.9% phenylpropanoid acids, Contemplated phenylpropanoid acids isolated from corn shoot or immature corn leaf extract are the following one or combination of more compounds including but not limited to cinnamic acid, coumaric acid, ferulic acid, phloretic acid, 7-methoxy-2,7-dihydroxy-2h-1,4-benzoxazin-3(4H)-one; 3-(4-Aminophenyl)-2-propenoic acid; (E)-form, 3-(2-Hydroxyphenyl)-2-propenoic acid; (E)-form, 3-(3-Hydroxyphenyl)-2-propenoic acid; (E)-form, 3-(4-Hydroxyphenyl)-2-propenoic acid; (E)-form, 3-(4-Hydroxyphenyl)-2-propenoic acid; (Z)-form, m-Cumaric acid, 2-Oxo-3-phenylpropanoic acid, 3-(4-Hydroxyphenyl)propanoic acid, 3-(2-Hydroxyphenyl)propanoic acid, 3-Hydroxy-3-phenylpropanoic acid; (ξ)-form, Ethyl cinnamate, 3-(4-Hydroxyphenyl)-2-propenoic acid; (Z)-form, Me ether, p-Methoxycinnamic acid, 3-(4-Hydroxyphenyl)-2-propenoic acid; (E)-form, Me ester, 3-(2-Hydroxyphenyl)-2-propenoic acid; (Z)-form, Me ether, Peplidoflorone D, α-(Hydroxyimino)benzenepropanoic acid, 3-Hydroxy-3-(2-methylphenyl)propenamide, 3-(3,4-Dihydroxyphenyl)-2-propenoic acid; (E)-form, α-(Hydroxyimino)benzenepropanoic acid, 3-Hydroxy-3-(2-methylphenyl)propenamide, 3-(3,4-Dihydroxyphenyl)-2-propenoic acid; (E)-form, Isocaffeic acid, Grevillic acid, 4-Hydroxyphenylpyruvic acid, Anthenobilic acid, 3-(3,5-Dihydroxyphenyl)-2-propenoic acid; (E)-form, 3-(4-Methoxyphenyl)propanoic acid, 2-Hydroxy-3-(4-hydroxyphenyl)propanoic acid; (R)-form, 2-Hydroxy-3-(4-hydroxyphenyl)propanoic acid; (S)-form, 2,4-Dihydroxyhydrocinnamic acid, 3,4-Dihydroxyhydrocinnamic acid, 2-Hydroxy-3-(4-hydroxyphenyl)propanoic acid; (ξ)-form, 2-Hydroxy-3-(2-hydroxyphenyl)propanoic acid; (±)-form, 2-Hydroxy-3-(2-hydroxyphenyl)propanoic acid; (S)-form, 3-(3,4-Methylenedioxyphenyl)-2-propenoic acid; (E)-form, 3-Formyl-4-hydroxycinnamic acid, 3-(4-Hydroxyphenyl)-2-propenoic acid; (E)-form, Me ether, Me ester, 3-(4-Hydroxyphenyl)-2-propenoic acid; (E)-form, Et ester, 3-(4-Hydroxyphenyl)-2-propenoic acid; (Z)-form, Me ether, Me ester, Ferulamide, 4-Hydroxy-3-methoxycinnamide, 3-(4-Hydroxy-3-methoxyphenyl)-2-propenoic acid; (E)-form, Isoferulic acid, o-Ferulic acid, 3-(3,4-Dihydroxyphenyl)-2-propenoic acid; (E)-form, Me ester, 2-Formyl-3-hydroxybenzenepropanoic acid, 3-(2,5-Dihydroxyphenyl)-2-propenoic acid; Me ester, 3-(4-Hydroxyphenyl)-3-oxopropanoic acid; Me ester, 3-Oxo-3-(4-methoxyphenyl)propanoic acid, 3-(4-Hydroxyphenyl)-2-oxopropanoic acid; Me ester, Graviquinone, 3-(3,5-Dihydroxyphenyl)-2-propenoic acid; (E)-form, Me ester, α-Oxyiminotyrosine, 3-Amino-3-(3,4-dihydroxyphenyl)propanoic acid; (R)-form, Danshensuan A, Vinyl caffeate, Methyl psilalate, 3-(4-Hydroxyphenyl)-2-propenoic acid; (E)-form, Me ether, Et ester, 3-(2-Carboxy-3-hydroxyphenyl)-2-propenoic acid; (E)-form, 3-(3,4-Dihydroxyphenyl)-2-propenoic acid; (E)-form, Et ester, 3-(4-Hydroxy-3-methoxyphenyl)-2-propenoic acid; (E)-form, Me ester, 3-(2,5-Dihydroxyphenyl)-2-propenoic acid; 2-Me ether, Me ester, 3-(3,4-Dihydroxyphenyl)-2-propenoic acid; (E)-form, 4′-Me ether, Me ester, 3-(4-Hydroxy-3-methoxyphenyl)-2-propenoic acid; (Z)-form, Me ester, 3-(2,4-Dihydroxyphenyl)-2-propenoic acid; (E)-form, 4-Me ether, Me ester, 3-(2,6-Dihydroxyphenyl)-2-propenoic acid; (E)-form, Di-Me ether, 3-(2-Formyl-3-hydroxyphenyl)propanoic acid; Me ester, 3-Nitro-p-coumaric acid, 3-(3-Carboxyphenyl)-2-hydroxypropanoic acid; (R)-form, 5-Hydroxyferulic acid, 3-(3,4-Dihydroxyphenyl)-2-oxopropanoic acid; (E)-enol-form, Me ester, 3-(3,4-Dihydroxyphenyl)-2-oxopropanoic acid; (Z)-enol-form, Me ester, 2,4-Dihydroxy-5-methoxycinnamic acid, 3,4-Dimethoxyhydrocinnamic acid, 3-(2,4-Dihydroxyphenyl)propanoic acid; 4-Me ether, Me ester, 2-Methoxy-3-(4-methoxyphenyl)propanoic acid, Latifolicinin B, 3-Nitrophloretic acid, 3-(3,4-Dihydroxyphenyl)glyceric acid, 3-(2,2-Dimethyl-2H-1-benzopyran-6-yl)-2-propenal, 4-(2,3-Butadienyloxy)cinnamic acid, Colpuchol, Methyl 3-(4-methoxy-2-vinylphenyl)propanoate, 3-Methoxy-2-(3,4-methylenedioxyphenyl)-2-propenoic acid; (E)-form, 3-Methoxy-4,5-methylenedioxycinnamic acid, 3-(4-Hydroxy-3-methoxyphenyl)-2-propenoic acid; (E)-form, Et ester, 3-(3,4-Dihydroxyphenyl)-2-propenoic acid; (E)-form, Di-Me ether, Me ester, 3-(3,4-Dihydroxyphenyl)-2-propenoic acid; (Z)-form, Di-Me ether, Me ester, 3-(2,5-Dihydroxyphenyl)-2-propenoic acid; 5-Et ether, Me ester, 3-[(4-Methylthio)phenyl]-2-butenoic acid; (E)-form, Me ester, Boehmerine, Sinapic acid, 3-(3,4,5-Trihydroxyphenyl)-2-propenoic acid; (Z)-form, 3,5-Di-Me ether, 3-(3,4-Dihydroxyphenyl)propanoic acid; Di-Me ether, Me ester, 3-(4-Hydroxy-3-nitrophenyl)propanoic acid; Me ester, 3-(2-Hydroxy-3,4-dimethoxyphenyl)propanoic acid, 3-(3,4-Dihydroxyphenyl)-2-hydroxypropanoic acid; (ξ)-form, Et ester, Taraxafolin, 3-(2,4,5-Trihydroxyphenyl)propanoic acid; 5-Me ether, Me ester, Pisoninol I, 2-Hydroxy-3-(4-hydroxy-3-nitrophenyl)propanoic acid, 2-Hydroxy-3-(4-hydroxy-3-nitrophenyl)propanoic acid; (ξ)-form, 3-(2,3-Dihydro-2-isopropenyl-5-benzofuranyl)-2-propenoic acid, 3-(2,2-Dimethyl-2H-1-benzopyran-6-yl)-2-propenoic acid; (E)-form, Drupacin, Wutaipyranol A, 1-[3-(2-Hydroxyphenyl)propanoyl]piperidine, 3-(3,4,5-Trihydroxyphenyl)-2-propenoic acid; (E)-form, 3,4-Methylene, 5-Me ether, Me ester, 3-Methoxy-2-(3,4-methylenedioxyphenyl)-2-propenoic acid; (E)-form, Me ester, Hydroxyacetic acid; 0-(4-Hydroxy-E-cinnamoyl), Me ester, Isobutyl caffeate, Cintriamide, 4,6-Dihydroxy-3-methyl-2-pyruvoylbenzoic acid, trans-Caffeoylglycolic acid, 3-(3-Carboxy-4-hydroxyphenyl)-2-methoxy-2-propenoic acid, 2,4,5-Trimethoxycinnamic acid, 3,4,5-Trimethoxycinnamic acid, 3-(2,4,5-Trihydroxyphenyl)-2-propenoic acid; (Z)-form, Tri-Me ether, Methyl sinapate, 3-(2,3,4-Trihydroxyphenyl)-2-propenoic acid; (Z)-form, Tri-Me ether, Methyl 4-ethoxy-3,5-dihydroxycinnamate, Microintegerrin A, Latifolicinin A, Propyl dihydroferulate, 3-(3,4-Dihydroxyphenyl)propanoic acid; Di-Me ether, Et ester, 3-Phenyl-2-propenoic acid; (E)-form, Benzyl ester, 3,4,5-Trimethoxydihydrocinnamic acid, 2,4,5-Trimethoxydihydrocinnamic acid, 3-O-Methyltaraxafolin, Chaetoisochorismin, o-Hydroxynitropapuline, 2-Chloro-3-(4-hydroxy-3-nitrophenyl)propanoic acid; (ξ)-form, Methyl 4-prenyloxycinnamate, Drupacin methyl ester, Z-Drupacin methyl ester, Parvifloral, 2,4-Dihydroxy-3-prenylcinnamic acid; (E)-form, 3,4-Dihydroxy-5-prenylcinnamic acid; (E)-form, Fimbriether C, 3-(3,4-Dihydro-3-hydroxy-2,2-dimethyl-2H-1-benzopyran-6-yl)-2-propenoic acid, 3-(4-Hydroxyphenyl)propanoic acid; 4-O-(3-Methyl-2-butenyl), Me ester, Wutaipyranol, 3-(4-Hydroxy-3-methoxyphenyl)-2-propenoic acid; (E)-form, Ac, Me ester, 3-(4-Hydroxyphenyl)propanoic acid; 4-Methylpentyl ester, 2,3-Dihydroxypropanoic acid; (ξ)-form, 2-O-(4-Hydroxy-E-cinnamoyl), 2,3-Dihydroxypropanoic acid; (ξ)-form, Hydroxy-Z-cinnamoyl), 2-(2-Formyl)-3-oxobutyl-4,6-dihydroxy-3-methylbenzoic acid, trans-Feruloylglycolic acid, Hydroxyacetic acid; 0-(3,4-Dihydroxy-E-cinnamoyl), Me ester, trans-Isoferuloylglycolic acid, 3-(2,4,5-Trihydroxyphenyl)-2-propenoic acid; (E)-form, Tri-Me ether, Me ester, Methyl 3,4,5-trimethoxycinnamate, 3-(3,4,5-Trihydroxyphenyl)-2-propenoic acid; (Z)-form, Tri-Me ether, Me ester, 3-(2,3,4-Trihydroxyphenyl)-2-propenoic acid; (E)-form, Tri-Me ether, Me ester, Ethyl sinapate, 3-(3,4,5-Trihydroxyphenyl)propanoic acid; 3′,4′-Methylene, 5′-Me ether, Et ester, 3-(4-Hydroxy-3,5-dimethoxyphenyl)oxiranecarboxylic acid; (2ξ,3ξ)-form, Me ester, Lavandunat, 3-(2,4,5-Trihydroxyphenyl)propanoic acid; Tri-Me ether, Me ester, 3-(3,4,5-Trihydroxyphenyl)propanoic acid; Tri-Me ether, Me ester, Benzyl (E)-p-coumarate, Benzyl (Z)-p-coumarate, 4-Hydroxy-3,5-dinitrohydrocinnamic acid, 3-Hydroxy-3-(3,4,5-trihydroxyphenyl)propanoic acid; (R)-form, 3′,5′-Di-Me ether, Me ester, 3-Hydroxy-3-(3,4,5-trihydroxyphenyl)propanoic acid; (S)-form, 3′,5′-Di-Me ether, Me ester or a combination thereof.

It is contemplated that benzoxazinoid is derived, obtained or selected from at least one of the following—alone or in combination with one another seedlings and all plant parts of corn, wheat, rye, rice, barley, oat, cereal, adlay, Sorghum plants and other plants such as Zea mays, Oryza species, Oryza sativa, Oryz glaberrima, Oryz australiensis, Oryz brachyantha, Secale cereale, Acanthus arboreus; Acanthus illicifolius, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, and Balsamocitrus paniculate; Peristrophe roxburghiana; Strobilanthes cusia; Scoparia dulcis, Lobelia chinensis, Leymus chinensis, a marine sponge Oceanapia sp. or a combination thereof.

It is contemplated that contemplated compositions enriched for one or more phenylpropanoid acids and benzoxazinoids, are derived, obtained, extracted, or selected from at least one of the following species of plants or extracts from at least one of the following species of plants, including but not limited to corn, wheat, rye, rice, barley, oat, cereal, adlay, Sorghum plants and other plants such as Zea mays, Oryza species, Oryza sativa, Oryz glaberrima, Oryz australiensis, Oryz brachyantha, Secale cereale, Acanthus arboreus; Acanthus ebracteatus, Acanthus illicifolius, Acanthus mollis, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, Agropyron repens, Blepharis edulis, Balsamocitrus paniculate; Peristrophe roxburghiana; Strobilanthes cusia; Lamium galeobdolon, Lobelia chinensis, Leymus chinensis, Aphelandra spp, Scoparia dulcis, Capparis sikkimensis ssp, a fungal species Monocillium sp, a marine sponge Oceanapia sp. or a combination thereof.

Contemplated compositions, enriched for one or more phenylpropanoid acids and benzoxazinoids, are obtained, derived or extracted from any suitable source or sources, including but not limited to seedlings, shoots, germinates from plant seeds, sprouts of geminated grains, immature leaves, mature leaves, whole plants, roots, seeds, flowers, stems, stem barks, root barks, silk, grain, hair roots of germinated grain, stem cells, cell culture tissues or any combination thereof corn, wheat, rye, rice, barley, oat, cereal, adlay, Sorghum plants and other species of plants including but not limited to Zea mays, Oryza species, Oryza sativa, Oryz glaberrima, Oryz australiensis, Oryz brachyantha, Secale cereale, Acanthus arboreus; Acanthus ebracteatus, Acanthus illicifolius, Acanthus mollis, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, Agropyron repens, Blepharis edulis, Balsamocitrus paniculate; Peristrophe roxburghiana; Strobilanthes cusia; Lamium galeobdolon, Lobelia chinensis, Leymus chinensis, Aphelandra spp, Scoparia dulcis, Capparis sikkimensis ssp, a fungal species Monocillium sp, a marine sponge Oceanapia sp. or a combination thereof.

Contemplated compositions enriched for one or more phenylpropanoid acids and one or more benzoxazinoids are synthesized, metabolized, biodegraded, bioconverted, biotransformed, biosynthesized from small carbon units, by transgenic microbial, by P450 enzymes, by glycotransferase enzyme or a combination of enzymes, by microbacteria.

Contemplated compositions are disclosed, wherein one or more phenylpropanoid acids and one or more benzoxazinoids in the composition establishes and regulates homeostasis of host stress hormone, cortisol, that leads to improved symptoms of chronically high cortisol, the symptoms including but not limited to anxiety, depression, fatigue, gastrointestinal upset like constipation, bloating, or diarrhea, headache, heart disease, high blood pressure, irritability, problems with memory and concentration, reproductive issues like low libido, erectile dysfunction, or irregular menstruation and ovulation, sleep difficulties, slow recovery from exercise, eating disorders and weight gain.

Contemplated compositions are disclosed, wherein one or more phenylpropanoid acids and one or more benzoxazinoids in the composition improves sleep quality by enhancing the deep sleep stage of sleep, increases total sleep time and deep sleep time, improves overall mental well-being measured by the Pittsburgh Sleep Quality Index (PSQI) and Profile of Mood States (POMS), provides positive mood support and enhances emotional well-being; maintains homeostasis of biomarkers—serotonin, melatonin, GABA in formulation in a mammal

Contemplated compositions are disclosed, wherein one or more phenylpropanoid acids and one or more benzoxazinoids in the composition prevents and treats sleep disorders including but not limited to insomnia, Hypersomnia, circadian rhythm disorders, shift work sleep disorder, non-24-hour sleep-wake disorder, periodic limb movement disorder, restless legs syndrome (RLS), sleep apnea, narcolepsy, Parasomnias, night terrors, sleepwalking, nightmares, sleep eating disorder. sleep hallucinations, sleep paralysis, sleep talking, REM sleep behavior disorder.

In the above and following descriptions, certain specific details are set forth in order to provide a thorough understanding of various embodiments of this disclosure. However, one skilled in the art will understand that the contemplated embodiments may be practiced without these details.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “and/or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to.”

Reference throughout this specification to “one embodiment” or “an embodiment” or “a contemplated embodiment” means that a particular feature, structure or characteristic described in connection with the embodiment is included in at least one embodiment of the present contemplated embodiments. Thus, the appearances of the phrases “in one embodiment” or “in an embodiment” or “a contemplated embodiment” in various places throughout this specification are not necessarily all referring to the same embodiment.

The term “prodrug” is also meant to include any covalently bonded carriers, which release the active compound of this disclosure in vivo when such prodrug is administered to a mammalian subject. Prodrugs of a compound of this disclosure may be prepared by modifying functional groups present in the compound of this disclosure in such a way that the modifications are cleaved, either in routine manipulation or in vivo, to the parent compound of this disclosure. Prodrugs include compounds of this disclosure wherein a hydroxy, amino or mercapto group is bonded to any group that, when the prodrug of the compound of this disclosure is administered to a mammalian subject, cleaves to form a free hydroxy, free amino or free mercapto group, respectively. Examples of prodrugs include acetate, formate and benzoate derivatives of alcohol or amide derivatives of amine functional groups in the compounds of this disclosure and the like.

“Stable compound” and “stable structure” are meant to indicate a compound that is sufficiently robust to survive isolation to a useful degree of purity from a reaction mixture, and formulation into an efficacious therapeutic agent.

“Biomarker(s)” or “marker(s)” component(s) or compound(s) are meant to indicate one or multiple indigenous chemical component(s) or compound(s) in the disclosed plant(s), plant extract(s), or combined composition(s) with 2-3 plant extracts that are utilized for controlling the quality, consistence, integrity, stability, and/or biological functions of the invented composition(s). Sometimes the quality marker compound(s) is(are) not bioactive compound(s) that directly related to the intended method of use.

“Mammal” includes humans and both domestic animals, such as laboratory animals or household pets (e.g., cats, dogs, swine, cattle, sheep, goats, horses, rabbits), and non-domestic animals, such as wildlife or the like.

“Optional” or “optionally” means that the subsequently described element, component, event or circumstances may or may not occur, and that the description includes instances where the element, component, event or circumstance occur and instances in which they do not. For example, “optionally substituted aryl” means that the aryl radical may or may not be substituted and that the description includes both substituted aryl radicals and aryl radicals having no substitution.

“Pharmaceutically or nutraceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically or nutraceutically acceptable salt” includes both acid and base addition salts. “Pharmaceutically or nutraceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically or nutraceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include the sodium, potassium, lithium, ammonium, calcium, magnesium, iron, zinc, copper, manganese, aluminum salts and the like. In certain embodiments, the inorganic salts are ammonium, sodium, potassium, calcium, or magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly useful organic bases are isopropylamine, diethylamine, ethanolamine, trimethylamine, dicyclohexylamine, choline and caffeine.

Often crystallizations produce a solvate of the compound of this disclosure. As used herein, the term “solvate” refers to an aggregate that comprises one or more molecules of a compound of this disclosure with one or more molecules of solvent. The solvent may be water, in which case the solvate may be a hydrate. Alternatively, the solvent may be an organic solvent. Thus, the compounds of the present contemplated embodiments may exist as a hydrate, including a monohydrate, dihydrate, hemihydrate, sesquihydrate, trihydrate, tetrahydrate and the like, as well as the corresponding solvated forms. The compound of this disclosure may be true solvates, while in other cases, the compound of this disclosure may merely retain adventitious water or be a mixture of water plus some adventitious solvent.

A “pharmaceutical composition” or “nutraceutical composition” refers to a formulation of a compound of this disclosure and a medium generally accepted in the art for the delivery of the biologically active compound to mammals, e.g., humans. For example, a pharmaceutical composition of the present disclosure may be formulated or used as a standalone composition, or as a component in a prescription drug, an over the counter (OTC) medicine, a botanical drug, an herbal medicine, a natural medicine, a homeopathic agent, or any other form of health care product reviewed and approved by a government agency. Exemplary nutraceutical compositions of the present disclosure may be formulated or used as a standalone composition, or as a nutritional or bioactive component in food, a functional food, a beverage, a bar, a food flavor, a medical food, a dietary supplement, or an herbal product. A medium generally accepted in the art includes all pharmaceutically or nutraceutically acceptable carriers, diluents or excipients therefor.

As mentioned earlier, “enriched for” refers to a plant extract or other preparation having at least a two-fold up to about a 1000-fold increase of one or more active compounds as compared to the amount of one or more active compounds found in the weight of the plant material or other source before extraction or other preparation. In certain embodiments, the weight of the plant material or other source before extraction or other preparation may be dry weight, wet weight, or a combination thereof.

As used herein, “major active ingredient” or “major active component” refers to one or more active compounds found in a plant extract or other preparation or enriched for in a plant extract or other preparation, which is capable of at least one biological activity. In certain embodiments, a major active ingredient of an enriched extract will be the one or more active compounds that were enriched in that extract. Generally, one or more major active components will impart, directly or indirectly, most (i.e., greater than 50%, or 20% or 10%, or 1% or 0.05%) of one or more measurable biological activities or effects as compared to other extract components. In certain embodiments, a major active ingredient may be a minor component by weight percentage of an extract (e.g., less than 50%, 25%, or 10% or 5% or 1% or 0.2% or 0.05% of the components contained in an extract) but still provide most of the desired biological activity. Any composition of this disclosure containing a major active ingredient may also contain minor active ingredients that may or may not contribute to the pharmaceutical or nutraceutical activity of the enriched composition, but not to the level of major active components, and minor active components alone may not be effective in the absence of a major active ingredient.

“Effective amount” or “therapeutically effective amount” refers to that amount of a compound or composition of this disclosure which, when administered to a mammal, such as a human, is sufficient to improve sleep disturbance, fragmentation, quality and efficiency through any one or combination of pathways such as 1) reduced nocturnal and/or diurnal plasma, urine or salivary cortisol level, 3) modulation of hypothalamus-pituitary-adrenal axis, 4) impact on non-rapid eye movement stages of sleep such as slow wave deep sleep, 5) activity on rapid eye movement stage of sleep, and 6) decreasing night time wakefulness.

The amount of a compound, an extract or a composition of this disclosure that constitutes a “therapeutically effective amount” will vary depending on the bioactive compound, or a standardized extract, or an ethanol extract or the biomarker for the condition being treated and its severity, the manner of administration, the duration of treatment, or the age of the subject to be treated, but can be determined routinely by one of ordinary skill in the art having regard to his own knowledge and to this disclosure. In certain embodiments, “effective amount” or “therapeutically effective amount” may be demonstrated as the quantity of bioactive compound or an extract over the body weight of a mammal (i.e., 0.001 mg/kg, 0.005 mg/kg, 0.01 mg/kg, or 0.1 mg/kg, or 1 mg/kg, or 5 mg/kg, or 10 mg/kg, or 20 mg/kg, or 50 mg/kg, or 100 mg/kg, or 200 mg/kg or 500 mg/kg or 1,000, g/kg). The human equivalent daily dosage can be extrapolated from the “effective amount” or “therapeutically effective amount” in an animal study by utilization of FDA guideline in consideration the difference of total body areas and body weights of animals and human.

“Dietary supplements” as used herein are a product that improves, promotes, increases, manages, controls, maintains, optimizes, modifies, reduces, inhibits, balance, a particular condition associated with a natural state or biological process, or a structural and functional integrity, an off-balanced or a compromised, or suppressed or impaired or overstimulated of a biological function or a phenotypic condition (i.e., are not used to diagnose, treat, mitigate, cure, or prevent disease). For example, with regard to sleep, dietary supplements may be used to modulate, maintain, manage, balance, suppress or stimulate any components of sleep and neuroendocrine system to yield an improved sleep quality, efficiency, and duration by correcting factors that would contribute to sleep disturbance, fragmentation, wakefulness, hyperactivity and/or disturbed HPA axis and increased cortisol level. In certain embodiments, dietary supplements are a special category of food, functional food, medical food and are not a drug.

“Treating” or “treatment” as used herein refers to the treatment of the disease or condition of interest in a mammal, such as a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving or modifying the disease or condition, i.e., causing regression of the disease or condition; or (iv) relieving the symptoms resulting from the disease or condition, (e.g., improving sleep quality and efficiency of patient diagnosed with a sleep disorder such as insomnia) without addressing the underlying disease or condition; (v) balancing the regulation of HPA axis homeostasis or changing the phenotype of the disease or condition

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians. A disease or condition may be acute such as insomnia; and may be chronic such as sleep disorder caused by aging. A compromised hypothalamic-pituitary-adrenal (HPA) axis function from off balance of homeostasis could cause a disease or a condition, or could make the Mammal more susceptible neurological disorders, or could lead to more acutely or chronically elevated cortisol directly or indirectly associated with sleep disorders.

As used herein, “statistical significance” refers to a p value of 0.05 or less when calculated using the Students t-test and indicates that it is unlikely that a particular event or result being measured has arisen by chance.

For the purposes of administration, the compounds of the present contemplated embodiments may be administered as a raw chemical or may be formulated as pharmaceutical or nutraceutical compositions. Pharmaceutical or nutraceutical compositions of the present contemplated embodiments comprise a compound of structures described in these contemplated embodiments and a pharmaceutically or nutraceutically acceptable carrier, diluent or excipient. The compound of structures described here are present in the composition in an amount which is effective to treat a particular disease or condition of interest—that is, in an amount sufficient to promote good sleep quality and efficiency as well as HPA axis homeostasis in general or any of the other associated indications described herein, and generally with acceptable toxicity to a patient.

Administration of the compounds or compositions of this disclosure, or their pharmaceutically or nutraceutically acceptable salts, in pure form or in an appropriate pharmaceutical or nutraceutical composition, can be carried out via any of the accepted modes of administration of agents for serving similar utilities. The pharmaceutical or nutraceutical compositions of this disclosure can be prepared by combining a compound of this disclosure with an appropriate pharmaceutically or nutraceutically acceptable carrier, diluent or excipient, and may be formulated into preparations in solid, semi solid, liquid or gaseous forms, such as tablets, capsules, powders, granules, ointments, solutions, beverage, suppositories, injections, inhalants, gels, creams, lotions, tinctures, sashay, ready to drink, masks, microspheres, and aerosols. Typical routes of administering such pharmaceutical or nutraceutical compositions include oral, topical, transdermal, inhalation, parenteral, sublingual, buccal, rectal, vaginal, or intranasal. The term parenteral as used herein includes subcutaneous injections, intravenous, intramuscular, intrasternal injection or infusion techniques.

Pharmaceutical or nutraceutical compositions of this disclosure are formulated so as to allow the active ingredients contained therein to be bioavailable upon administration of the composition to a patient. Compositions that will be administered to a subject or patient or a mammal take the form of one or more dosage units, where for example, a tablet may be a single dosage unit, and a container of a compound or an extract or a composition of 2-3 plant extracts of this disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of this disclosure, or a pharmaceutically or nutraceutically acceptable salt thereof, for treatment of a disease or condition of interest in accordance with the teachings of this contemplated embodiments.

A pharmaceutical or nutraceutical composition of this disclosure may be in the form of a solid or liquid. In one aspect, the carrier(s) are particulate, so that the compositions are, for example, in tablet or in powder form. The carrier(s) may be liquid, with the compositions being, for example, oral syrup, injectable liquid or an aerosol, which is useful in, for example, inhalatory administration.

When intended for oral administration, the pharmaceutical or nutraceutical composition is in either solid or liquid form, where semi solid, semi liquid, suspension and gel forms are included within the forms considered herein as either solid or liquid.

As a solid composition for oral administration, the pharmaceutical or nutraceutical composition may be formulated into a powder, granule, compressed tablet, pill, capsule, chewing gum, sashay, wafer, bar, or like form. Such a solid composition will typically contain one or more inert diluents or edible carriers. In addition, one or more of the following may be present: binders such as carboxymethylcellulose, ethyl cellulose, cyclodextrin, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical or nutraceutical composition is in the form of a capsule, for example, a gelatin capsule, it may contain, in addition to materials of the above type, a liquid carrier such as polyethylene glycol or oil.

The pharmaceutical or nutraceutical composition may be in the form of a liquid, for example, an elixir, tincture, syrup, solution, emulsion or suspension. The liquid may be for oral administration or for delivery by injection, as two examples. When intended for oral administration, a useful composition contains, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical or nutraceutical compositions of this disclosure, whether they be solutions, suspensions or other like form, may include one or more of the following adjuvants: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a generally useful adjuvant. An injectable pharmaceutical or nutraceutical composition is sterile.

A liquid pharmaceutical or nutraceutical composition of this disclosure intended for either parenteral or oral administration should contain an amount of a compound of this disclosure such that a suitable dosage will be obtained.

The pharmaceutical or nutraceutical composition of this disclosure may be intended for topical administration, in which case the carrier may suitably comprise a solution, emulsion, cream, lotion, ointment, or gel base or a patch. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical or nutraceutical composition for topical administration. If intended for transdermal administration, the composition may include a transdermal patch or iontophoresis device.

The pharmaceutical or nutraceutical composition of this disclosure may be intended for rectal administration, in the form, for example, of a suppository, which will melt in the rectum and release the drug. The composition for rectal administration may contain an oleaginous base as a suitable nonirritating excipient. Such bases include lanolin, cocoa butter and polyethylene glycol.

The pharmaceutical or nutraceutical composition of this disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical or nutraceutical composition of this disclosure in solid or liquid form may include an agent that binds to the compound of this disclosure and thereby assists in the delivery of the compound. Suitable agents that may act in this capacity include a monoclonal or polyclonal antibody, a protein or a liposome.

The pharmaceutical or nutraceutical composition of this disclosure in solid or liquid form may include reducing the size of a particle to, for example, improve bioavailability. The size of a powder, granule, particle, microsphere, or the like in a composition, with or without an excipient, can be macro (e.g., visible to the eye or at least 100 μm in size), micro (e.g., may range from about 100 μm to about 100 nm in size), nano (e.g., may no more than 100 nm in size), and any size in between or any combination thereof to improve size and bulk density.

The pharmaceutical or nutraceutical composition of this disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of this disclosure may be delivered in single phase, bi phasic, or tri phasic systems in order to deliver the active ingredient(s). Delivery of the aerosol includes the necessary container, activators, valves, sub-containers, and the like, which together may form a kit. One skilled in the art, without undue experimentation, may determine the most appropriate aerosol(s).

The pharmaceutical or nutraceutical compositions of this disclosure may be prepared by methodology well known in the pharmaceutical or nutraceutical art. For example, a pharmaceutical or nutraceutical composition intended to be administered by injection can be prepared by combining a compound of this disclosure with sterile, distilled, deionized water so as to form a solution. A surfactant may be added to facilitate the formation of a homogeneous solution or suspension. Surfactants are compounds that non covalently interact with the compound of this disclosure so as to facilitate dissolution or homogeneous suspension of the compound in the aqueous delivery system.

The compounds of this disclosure, or their pharmaceutically or nutraceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of this disclosure, or pharmaceutically or nutraceutically acceptable derivatives thereof, may also be administered simultaneously with, prior to, or after administration of food, water and one or more other therapeutic agents. Such combination therapy includes administration of a single pharmaceutical or nutraceutical dosage formulation which contains a compound or an extract or a composition with 2-3 plant extracts of this disclosure and one or more additional active agents, as well as administration of the compound or an extract or a composition with 2-3 plant extracts of this disclosure and each active agent in its own separate pharmaceutical or nutraceutical dosage formulation. For example, a compound or an extract or a composition with 2-3 plant extracts of this disclosure and another active agent can be administered to the patient together in a single oral dosage composition, such as a tablet or capsule, or each agent can be administered in separate oral dosage formulations. Where separate dosage formulations are used, the compounds of this disclosure and one or more additional active agents can be administered at essentially the same time, i.e., concurrently, or at separately staggered times, i.e., sequentially; combination therapy is understood to include all these regimens.

It is understood that in the present description, combinations of substituents or variables of the depicted formulae are permissible only if such contributions result in stable compounds.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include C(O) R″ (where R″ is alkyl, aryl or arylalkyl), p methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of these contemplated embodiments may not possess pharmacological activity as such, they may be administered to a mammal and thereafter metabolized in the body to form compounds of this disclosure which are pharmacologically active. Such derivatives may therefore be described as “prodrugs”. All prodrugs of compounds of these contemplated embodiments are included within the scope of this disclosure.

Furthermore, all compounds or extracts of this disclosure which exist in free base or acid form can be converted to their pharmaceutically or nutraceutically acceptable salts by treatment with the appropriate inorganic or organic base or acid by methods known to one skilled in the art. Salts of the compounds of this disclosure can be converted to their free base or acid form by standard techniques.

Contemplated compounds, medicinal compositions and compositions may comprise or additionally comprise or consist of at least one active ingredient. In some embodiments, at least one bioactive ingredient may comprise or consist of plant powder or plant extract of or the like.

In any of the aforementioned embodiments, the standardized extract comprising mixtures of water or alcohol or supercritical fluid extracts of a composition derived from enriched for one or more phenylpropanoid acids and benzoxazinoids is mixed at a particular ratio by weight. In certain embodiments, the ratio (by weight) of at least one or more benzoxazinoids mixed with one or more phenylpropanoid acids in ranges from about 0.05:99.95 to about 99.95:0.05. Similar ranges apply when more than two extracts or compounds (e.g., three, four, five) are used. Exemplary ratios include 0.05:99.95, 0.1:99.9, 0.15:99.85, 0.2:99.8, 0.25:99.75, 0.3:99.7, 0.4:99.6, 0.5:99.5, 0.6:99.4, 1:99, 2:98, 3:97, 4:96, 5:95, 6:94, 7:93, 8:92, 9:91, 10:90, 15:85, 20:80, 25:75, 30:70, 50:50, 60:40, 70:30, 80:20, 90:10, 95:5, 99:1, 99.5:0.5, 99.9:0.1, 99.95:0.05. In further embodiments, the disclosed standardized extract of a composition derived from enriched for one or more phenylpropanoid acids and benzoxazinoids has been combined into a composition called UP165 as an example but not limited to be enriched to or standardized at 0.2% of a quality marker compound: 6-MBOA. In further embodiments, such enrichment or standardization of Zea mays extract is mixed one or more phenylpropanoid acids and benzoxazinoids either naturally isolated or artificially synthesized with equivalent chemical structure(s) of natural compound(s).

In further embodiments, such enrichment or standardization of Zea mays extract, were evaluated on in vitro, and/or ex vivo and/or in vivo models for advantage/disadvantage and unexpected synergy/antagonism of the perceived biological functions and effective adjustments of the homeostasis of neuroendocrine function and reduction of cortisol to yield improved sleep quality in human clinical trial. The best standardization with specific blending ratio of individual compounds were selected based on unexpected synergy measured on the in vitro, and/or ex vivo and/or in vivo models and potential enhancement of ADME of these compounds maximize the biological outputs.

In certain examples, a composition of this disclosure may be formulated to further comprise a pharmaceutically or nutraceutically acceptable carrier, diluent, or excipient, wherein the pharmaceutical or nutraceutical formulation comprises from about 0.01 or 0.05 weight percent (wt %), or or 0.2% or, 0.5 weight percent (wt %), or 5%, or 25% to about 95 wt % of active or major active, or biomarker compound(s) of an extract mixture. In any of the aforementioned formulations, a composition of this disclosure is formulated as a tablet, hard capsule, soft gel capsule, powder, or granule.

Also contemplated herein are agents of the disclosed compounds. Such products may result from, for example, the oxidation, reduction, hydrolysis, amidation, esterification, and the like of the administered compound, primarily due to enzymatic processes. Accordingly, contemplated compounds are those produced by a process comprising administering a contemplated compound or composition to a mammal for a period of time sufficient to yield a metabolic product thereof. Such products are typically identified by administering a radiolabeled or not radiolabeled compound of this disclosure in a detectable dose to an animal, such as rat, mouse, guinea pig, dog, cat, pig, sheep, horse, monkey, or human, allowing sufficient time for metabolism to occur, and then isolating its conversion products from the urine, blood or other biological samples.

Contemplated compounds, medicinal compositions and compositions may comprise or additionally comprise or consist of at least one pharmaceutically or nutraceutically or cosmetically acceptable carrier, diluent or excipient. As used herein, the phrase “pharmaceutically or nutraceutically or cosmetically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Contemplated compounds, medicinal compositions and compositions may comprise or additionally comprise or consist of at least one pharmaceutically or nutraceutically or cosmetically acceptable salt. As used herein, the phrase “pharmaceutically or nutraceutically or cosmetically acceptable salt” includes both acid addition and base addition salts.

In any of the aforementioned embodiments, the compositions comprising the enriched or standardized immature corn leaf extract and combination of one or more bioactive extracts or compounds to complement or boost the effects for regulating homeostasis of cortisol and improving sleep quality that may be present at certain percentage levels or ratios. In certain embodiments, the contemplated embodiments can include a composition derived from enriched for one or more phenylpropanoid acids and benzoxazinoids or standardized immature corn leaf extract as one of biomarker compounds and/or extracts from about 0.01% to about 99.9% phenylpropanoid acids and benzoxazinoids and/or its like compounds, or derivatives or precursors that can be isolated from natural sources or synthesized.

Natural bioactive compounds or extracts in combination with enriched or standardized phenylpropanoid acids and benzoxazinoids in immature corn leaf or corn shoot extract disclosed in current contemplated embodiments contain molecules that modulate the HPA axis and normalize the cortisol level for homeostatic feedback that will lead to an improved sleep quality and efficiency. Those natural compounds to be combined further with the current contemplated embodiments disclosed composition containing phenylpropanoid acids and benzoxazinoids in nutraceutical products include but not limited to Melatonin, Magnesium, gamma aminobutyric acid (GABA), vitamin B1, B2, B3, B6, B12, Pyridoxine, methylcobalamin, Niacinamide, Folic acid, Ascorbic acid, vitamin C, Vitamin D and E, Zinc, omega-3 fatty acid, Glycine, Glutamine, arginine, Tryptophan, L-theanine, 5-Hydroxytryptophan (5-HTP), SAMe, chlorella, Magnolol, Honokiol, Taurine, Boron, Branched-Chain amino acids (BCAA), phospholipids, phosphatidylserine, phosphatidic acid, theaflavin, rosmarinic acid, catechin, epicatechin, conjugated catechins such as EGCG, ECG, epigallocatechin etc. baicalein, baicalin, Oroxylin, Wogonin, Kaempferol, genistein, quercetin, Butein, Betaine, Luteolin, chrysin, Apigenin, curcumin, resveratrol, glomeratose A, 6-shogaol, gingerol, berberine, piperine,

The plant species that can be in combination with the current contemplated embodiments disclosed composition containing phenylpropanoid acids and benzoxazinoids in nutraceutical products to modulate the HPA axis and normalize the cortisol level for a homeostatic feedback that will lead to an improved sleep quality and efficiency including but not limited to Valerian roots, Valeriana officinalis, Ginkgo biloba, Kava kava, Lavender, Passionflower (Passiflora incarnata or maypop), Chamomile flower, Hops, chlorella, Humulus lupulus, Hibiscus sabdariffa, St. John's Worth, Calif Griffonia simplicifolia, Fermented milks, fish oil, Rhodiola rosea, Lotus seed, Lotus seed germ, Oryza sativa, Zea mays, Ziziphus jujuba, Schisandra chinensis, Magnolia officinalis, Astragalus membranaceus, Ganoderma lucidum, Echinacea purpurea, Echinacea angustifolia, Poria cocos Wolf, Wolfiporia extensa, Withania somnifera, Bupleurum falcatum, Glycyrrhiza spp, Panax quinquefolium, Panax ginseng C. A. Meyer, Korea red ginseng, Eurycoma longifolia (Malaysian ginseng) Lentinula edodes (shiitake), Inonotus obliquus (Chaga mushroom).

In some embodiments, the current contemplated embodiments disclosed composition containing phenylpropanoid acids and benzoxazinoids can be isolated from plant and/or marine sources, for example, from those plants included in the Examples and elsewhere throughout the present application. Suitable plant parts for isolation of the compounds include shoots, sprouts, leaves, immature leaf, bark, trunk, trunk bark, stem, stem bark, twigs, tubers, root, rhizome, root bark, bark surface, young shoots, seeds, fruits, seedlings, root hairs, androecium, gynoecium, calyx, stamen, petal, sepal, carpel (pistil), flowers, or any combination thereof. In some related embodiments, the phenylpropanoid acids and benzoxazinoids compounds or extracts are isolated from plant sources and synthetically made or modified to contain any of the recited substituents. In this regard, synthetic modification of the compound isolated from plants can be accomplished using any number of techniques which are known in the art and are well within the knowledge of one of ordinary skill in the art.

### EXAMPLES

### Example 1: Preparation of Organic Extracts from Corn Leaves (Zea mays)

Dried ground immature corn leaf powder (Zea mays) (10 g) loaded into two 100 ml stainless steel tube and extracted twice with different organic solvents, including dichloromethane, methanol, ethanol, acetone, petroleum and ethyl acetate using an ASE 300 automatic extractor at 80° C. and pressure 1500 psi. The extract solution is automatically filtered and collected. The organic extract solution is evaporated with rotary evaporator to give crude organic extracts as listed in the Table 1.

A composition derived from corn leaf extract enriched for one or more phenylpropanoid acids and benzoxazinoids (UP165) was produced as 70% Ethanol/30% water extract of ground immature corn leaf powder at 70-90° C. and standardized with no less than 0.2% 6-MBOA that is isolated from natural sources or synthesized.

Similar results were obtained using the same procedure, but with the organic solvent being replaced with methanol or ethanol to provide a methanol extract (ME) or ethanol extract (EE), methanol:H₂O (7:3) extracts, methanol:H₂O (1:1) extracts, methanol:H₂O (3:7) ethanol:H₂O (7:3) extracts, ethanol:H₂O (1:1) extracts, ethanol:H₂O (3:7) extracts and water extracts respectively.

TABLE 1
Extraction yield of corn leaf extracts

| Solvent | DCM | MeOH | EtOH | Acetone | EtOAc |
|---|---|---|---|---|---|
| Extraction yield (%) | 2.9% | 9.6% | 6.1% | 2.8% | 2.4% |

### Example 2: 6-MBOA Quantification in Corn Leaf Extracts

The immature corn leaf extracts (10 mg/mL) were analyzed on a Hitachi HPLC/PDA system with a C18 reversed-phase column (Phenomenex, Luna 5 μm, 150 mm×4.6 mm), eluted with 0.2% formic acid in H₂O and acetonitrile solvent system at a flow rate of 1 mL/min with UV detection at a wavelength of 286 nm with an injection volume of 10 μL against pure 6-MBOA (543551, Sigma-Aldrich) as external reference standard, prepared at a concentration of 0.2 mg/mL with the same injection volume. The 6-MBOA contents in plant extracts were determined in a range of 0.09-0.3% in the extracts obtained by different solvents including but not limited to methanol, ethanol, dichloromethane (DCM), acetone, ethyl acetate.

TABLE 2
6-MBOA content in corn leaf extracts

| | A composition | | | | | |
|---|---|---|---|---|---|---|
| | derived from | | | | | |
| | enriched for one | | | | | |
| | or more | | | | | |
| | phenylpropanoid | | | | | |
| | acids and | | | | | |
| | benzoxazinoids | | | | | |
| Extract | UP165 | DCM | MeOH | EtOH | Acetone | EtOAc |
| 6-MBOA content | 0.2% | 0.22% | 0.09% | 0.15% | 0.13% | 0.3% |
| (%) | | | | | | |

### Example 3: Solvent Partition and Fractionation of a Composition Derived from Enriched for One or More Phenylpropanoid Acids and Benzoxazinoids

A composition derived from enriched for one or more phenylpropanoid acids and benzoxazinoids (FP041019-01, 1 g) was partitioned between ethyl acetate (20 mL) and water (30 mL) for three times. The combined ethyl acetate solution was freed from solution by vacuum to give ethyl acetate s extract (EA) 31 mg. The aqueous layer was further extracted with butanol (20 mL) for three times to give butanol extract (BU) 60.6 mg. The remaining aqueous layer was freeze-dried to give aqueous extract (WA) 926.7 mg.

Fractionation of a composition derived from enriched for one or more phenylpropanoid acids and benzoxazinoids (FP041019-01, 5 g) was carried out by normal phase chromatography with silica column (Biotage Sfar Silica D Duo-100 g) using a Biotage Selekt system with a gradient mobile phase of 50:50 EtOAc:hexane, and increased to 100% ethyl acetate in 3 column volume, followed from 100% ethyl acetate to 100% methanol in 4 column volume at a flow rate of 120 ml/min, ended with a final wash of 100% methanol for two more column volume. The eluent was combined to 8 fractions based on a broadband wavelength UV detection and tested in melatonin receptor binding assay. Among 8 fractions, Fraction 04 and 05 showed highest inhibition against both MT1 and MT2 receptor. For partition samples, EA fraction showed the strongest inhibition with 77% inhibition at 50 ug/mL against MT1, while 91° A inhibition against MT2 at the same concentration.

TABLE 3
Samples tested for MT1 and MT2 receptor binding

| | MT1 (% inhibition) | | MT2 (% inhibition) | |
|---|---|---|---|---|
| Sample Name | 250 μg/mL | 50 μg/mL | 250 μg/mL | 50 μg/mL |
| M-01-02 | 86 | 65 | 97 | 83 |
| M-03 | 98 | 81 | 99 | 91 |
| M-04 | 94 | 68 | 99 | 90 |
| M-05 | 63 | 30 | 89 | 61 |
| M-06 | 49 | 18 | 77 | 45 |
| M-07 | 46 | 20 | 76 | 46 |
| M-08 | 49 | 22 | 72 | 38 |
| M-EA | 95 | 77 | 100 | 91 |
| M-BU | 81 | 39 | 95 | 75 |
| M-WA | 19 | 13 | 16 | 14 |

### Example 4: Evaluation the Chemical Profile of Corn Leaf Extracts from Different Heights of Immature Leaves of Zea mays

The corn (Zea mays) seeds were planned in the prepared soil to grow the plant under the standard agriculture practice. Corn leaves at different growth heights of the plant were harvested from 5 days after the seed germination to 45 days of germination as the following heights of the immature plant: #1. 110 cm; #2. 90 cm; #3. 72 cm; #4. 52 cm; and #5. 35 cm and #6 25 cm. The harvested immature corn shoots or leaves from above different heights of Zea mays plant were extracted with 70% Ethanol/30% water, and the solvents were evaporated under vacuum to yield dried extracts from different heights of the plant. The chemical profiles of those corn leaf extracts were analyzed with LC/MS/PDA and proton NMR against A contemplated immature corn leaf extract (UP165, Lot #FP041019-01) with 6-MBOA contents were quantified according to the method in Example 2. The extraction yields, 6-MBOA contents and chemical profile comparison were summarized to determine the most economic heights of the plant to be harvested for making a standardized composition derived from enriched for one or more phenylpropanoid acids and benzoxazinoids, in which those compounds can be synthesized or isolated from immature corn leaf including but not limited to 6-Methoxy-2-benzoxazolol (MBOA); 2-Benzoxazolol (BOA); 4-Methylbenzoxazole; 2,4-Dimethylbenzoxazole; 2,6-Dimethylbenzoxazole; 2,6-Benzoxazolediol; 2,4-Benzoxazolediol; 4-Acetyl-2(3H)-benzoxazolone; 6-Methoxy-N-methyl-2(3H)-benzoxazolone; 3-Hydroxy-6-methoxy-2-benzoxazolin-2(3H)-one; Hydroxy-6,7-dimethoxybenzoxazole; 5,6-Dimethoxy-2-benzoxazolinone; 3,6-Dimethoxybenzoxazolin-2(3H)-one; 5-Chloro-6-methoxy-2-benzoxazolinone; Trehalamine or a combination thereof.

TABLE 4
6-MBOA content in corn shoot or immature
corn leaf extracts with different heights

| | Corn shoot or immature | 6-MBOA content |
|---|---|---|
| Sample NO. | plant height | in extract |
| #1 | 110 cm | 0.106% |
| #2 | 90 cm | 0.120% |
| #3 | 72 cm | 0.168% |
| #4 | 52 cm | 0.215% |
| #5 | 35 cm | 0.262% |
| #6 | 25 cm | 0.314% |

### Example 5: Evaluation 6-Mboa Contents and Composition Profiles with Enriched for One or More Phenylpropanoid Acids and Benzoxazinoids in Different Crops Germinated after 10 Days

Plant seeds of 6 crop species were planted in the prepared soil to grow the plant under the standard agriculture practice grown in Texas in early Spring and whole plant shoots were harvested 10 days after germination. The ground and dried plant shoot powder was extracted with methanol to give methanol extract for each plant. Extracts were prepared at 10 mg/mL concentration and analyzed by ACQUITY UPLC-I-class Xevo G2-XS-QTof system for composition profiles with enriched for one or more phenylpropanoid acids and benzoxazinoids. 6-MBOA was also quantified at 0.66% in corn shoot extract and 0.12% in the corn shoot powder; while 0.19% in wheat shoot extract and 0.03% in the wheatgrass powder; 0.0081% in rye shoot extract and 0.0018% in the rye grass powder. 6-MBOA was not detected in barley, oats, and buckwheat extracts in this study.

The ground and dried corn young shoot and wheatgrass powder were also extracted with water and water with 2% acetic acid. Extracts were prepared at 10 mg/mL concentration and analyzed by ACQUITY UPLC-I-class Xevo G2-XS-QTof system to determine the profiles of enriched one or more phenylpropanoid acids and benzoxazinoids as well as the content for 6-MBOA.

Coix lacryma-jobi L seed was directly extracted with methanol and analyzed by ACQUITY UPLC-I-class Xevo G2-XS-QTof system at a concentration of 50 mg/mL concentration. The profiles of enriched one or more phenylpropanoid acids and benzoxazinoids as well as the 6-MBOA was determined at 0.00226% in the coix seed powder.

TABLE 5
6-MBOA contents in shoot extracts of different
plants 10-days after gemination of those crops

| Sample | | Common | | 6-MBOA in | 6-MBOA in |
|---|---|---|---|---|---|
| Code | Plant species | name | Extract ID | extract | plant |
| L0879 | Zea mays | Corn | L0879-ME | 0.66% | 0.12% |
| L0880 | Hordeum vulgare | Barley | L0880-ME | Not detected | Notdetected |
| L0881 | Avena sativa | Oats | L0881-ME | Not detected | Notdetected |
| L0882 | Triticum aestivum | Wheat | L0882-ME | 0.19% | 0.03% |
| L0883 | Secale cereale | Rye | L0883-ME | 0.0081% | 0.0018% |
| L0884 | Fagopyrum esculentum | Buckwheat | L0884-ME | Not detected | Notdetected |

TABLE 6
6-MBOA contents in corn and wheat extracts

| | Sample | | Extraction | 6-MBOA |
|---|---|---|---|---|
| Sample code | description | Extract (g) | Yield | in extract |
| L0879-ME | Methanol extract | 3.6849 | 18.42% | 0.66% |
| L0879-AE | Water extract | 3.8667 | 19.33% | 0.41% |
| L0879-AEA | 2% acetic acid | 2.86841 | 14.34% | 0.48% |
| | extract | | | |
| L0882-ME | Methanol extract | 3.2152 | 16.08% | 0.19% |
| L0882-AE | Water extract | 4.4838 | 22.42% | 0.11% |
| L0882-AEA | 2% acetic acid | 4.0747 | 20.37% | 0.07% |
| | extract | | | |

### Example 6: Bioassay-Guided Isolation and Identification

The immature corn leaf extracts (200 g) were partitioned between ethyl acetate and water three times to give EA fraction (6.42 g). The EA fraction was by subjected to reversed phase chromatography by Biotage SNAP cartridge (KP-C18) with a gradient elution of mixture of methanol/water at a flow rate of 20 mL/min. 88 fractions were collected and combined to give 11 subfractions. 11 samples were submitted for melatonin receptor binding assay (See table 7). EA-F4 and EA-F5 showed the strongest activity against MT2.

The EA-F5 was further separated by prep-HPLC on a Phenomenex Luna C18 column (250×30 mm, 10 μm) eluted with mobile phase acetonitrile and water containing 0.1% FA. 4 pure compounds were isolated from EA-F5 and identified as 3 phenolic acids including 4-hydroxycinnamic acid, ferulic acid, 3-methoxy-coumaric acid and one benzoxazinoid 2-Hydroxy-7-methoxy-2H-1,4-benzoxazin-3(4H)-one (HMBOA). None of the isolated compounds showed good inhibition at the same concentration 2.5 μg/mL compared to the original 51% inhibition of EA-F5 against MT2 receptor binding.

Another fraction EA-F4 was also fractionated by prep-HPLC on a Phenomenex Luna C18 column (250×30 mm, 10 μm) eluted with mobile phase acetonitrile and water containing 0.1% FA. 13 fractions were collected from EA-F4. EA4-F2 showed very potent MT2 receptor inhibition with 82.5% at 2.5 μg/mL, and 50.3% at 0.5 μg/mL. Two pure benzoxazinoid glycosides were isolated from this active fraction and were identified as 2-(β-D-Glucopyranosyloxy)-4,7-dimethoxy-2H-1,4-benzoxazin-3(4H)-one (MW387) and 2-(β-D-Glucopyranosyloxy)-7-methoxy-2H-1,4-benzoxazin-3(4H)-one (HMBOA-Glc, MW357). Neither compound show any inhibition against either MT1 or MT2 receptor in a concentration range from 8 ug/mL to 0.015625 μg/mL.

Phloretic acid was isolated as major component from two active fractions EA4-F7 and EA4-F8, showed 69% MT2 inhibition at 5 μg/mL concentration, much better inhibition compared to other cinnamic acids isolated, compatible to crude fraction EA4-F7 with 45.8% inhibition, and EA4-F8 48% inhibition at 2.5 μg/mL concentration.

The MT2 receptor binding inhibition is either lost or reduced after purification, strongly indicating the potent efficacy is not from single type of actives but coming from a combination of two types of components, phenolic acids, specifically phenylpropanoid acids, and benzoxazinoid glycosides. EA-F5, with IC₅₀ close to 2.5 μg/mL, which contains phenolic acids and benzoxazinoid glycosides in a ratio of 2:1 based on proton NMR analysis. EA-F4-2 showed potent inhibition with IC₅₀ around 0.5 μg/mL concentration, containing phenolic acids and benzoxazinoids in a ratio of 1:2 based on the proton NMR analysis.

TABLE 7
EA fractions tested for MT2 receptor binding

| | MT2 (% inhibition) | | | |
|---|---|---|---|---|
| Sample Name | 50 μg/mL | 25 μg/mL | 10 μg/mL | 2.5 μg/mL |
| EA-F2 | 78 | 61 | 40 | 23 |
| EA-F3 | 88 | 80 | 67 | 43 |
| EA-F4 | 93 | 90 | 87 | 68 |
| EA-F5 | 91 | 87 | 77 | 51 |
| EA-F6 | 89 | 81 | 70 | 45 |
| EA-F7 | 99 | 95 | 84 | 61 |
| EA-F8 | 97 | 95 | 87 | 63 |
| EA-F9 | 92 | 87 | 78 | 49 |
| EA-F10 | 91 | 85 | 62 | 36 |
| EA-F11 | 87 | 78 | 50 | 24 |
| EA-F12 | 71 | 50 | 22 | 13 |

TABLE 8
Subfractions from EA-F4 tested for MT2 receptor binding

| | 2.5 μg/mL | 0.5 μg/mL |
|---|---|---|
| EA4-F2 | 82.5 | 50.3 |
| EA4-F3 | 59.2 | 29.1 |
| EA4-F4 | 33.3 | 9.8 |
| EA4-F5 | not tested | not tested |
| EA4-F6 | 59.7 | 26.7 |
| EA4-F7 | 45.9 | 14.8 |
| EA4-F8 | 48 | 24.7 |
| EA4-F9 | not tested | not tested |
| EA4-F10 | not tested | not tested |
| EA4-F11 | 16.6 | 6.6 |
| EA4-F12 | 25.7 | 6.3 |
| EA4-F13 | 15 | 8.3 |

TABLE 9
Compounds isolated from fractions of EA-F5
and EA4 tested for MT2 receptor binding

| | Molecular | 25 | 5 | 2.5 | 0.5 |
|---|---|---|---|---|---|
| Compound Name | weight | μg/mL | μg/mL | μg/mL | μg/mL |
| p-coumaric acid | 164 | | | 6.7 | 0.5 |
| Ferulic acid | 194 | | | 1.6 | 2.6 |
| 3-methoxy- | 196 | | | 22.9 | 8.9 |
| hydrocoumaric | | | | | |
| acid | | | | | |
| HMBOA | 195 | | | 28 | 3.9 |
| Phloretic acid | 166 | 94 | 69 | | |

TABLE 10
Benzoxazinoids isolated from EA4-F2 tested
in MT1 and MT2 receptor binding

| | |
|---|---|
| MW357 | MW387 |

| Conc. μg/mL | MT1 | MT2 | MT1 | MT2 |
|---|---|---|---|---|
| 8 | 0 | 3 | 3 | 4 |
| 4 | −5 | 5 | −5 | 3 |
| 2 | −4 | 10 | −1 | 0 |
| 1 | −2 | 3 | 1 | −2 |
| 0.5 | −5 | 4 | 6 | 7 |
| 0.25 | 0 | 5 | 6 | −3 |
| 0.125 | −1 | −6 | −2 | −6 |
| 0.0625 | −4 | 1 | −6 | −2 |
| 0.03125 | 1 | 2 | −3 | −6 |
| 0.015625 | 3 | 4 | 2 | −4 |

### Example 7: Effect of a Composition Derived from Enriched for One or More Phenylpropanoid Acids and Benzoxazinoids on Melatonin Receptor (MT1 and MT2) Binding Assay

Human recombinant melatonin receptors (MT1 and MT2) were expressed in CHO-K1 cells and resuspended in buffer. The membranes containing recombinant receptors were incubated with test compounds in combination with 0.05 nM [125I]2-lodomelatonin for 180 minutes at 25° C. Membranes were immobilized on filters before being washed and the [125I]2-Iodomelatonin was counted. Test compounds that bound to MT1 or MT2 in the same binding site as melatonin displaced [125I]2-lodomelatonin, reducing the count. 1 μM 6-chloromelatonin was used to estimate non-specific binding, as its affinity for MT1 and MT2 is lower than 2-lodomelatonin. 0.00021 μM Melatonin was used as a positive control to displace [125I]2-Iodomelatonin (Paul et al., 1999; Beresford et al., 1998).

A composition derived from enriched for one or more phenylpropanoid acids and benzoxazinoids (UP165, Lot #FP041019-01) was tested in duplicate at 12 concentrations for MT1 binding (0.78, 1.56, 3.12, 6.25, 12.5, 25, 50, 100, 200, 400, 800, and 1600 μg/mL), and 6-MBOA was tested in duplicate at 10 concentrations (3.2 nM, 6.4 nM, 13 nM, 0.025 μM, 0.051 μM, 0.102 μM, 0.204 μM, 0.407 μM, 0.815 μM, and 1.63 μM), which were equivalent to the 6-MBOA content present in 0.19, 0.39, 0.78, 1.56, 3.12, 6.25, 12.5, 25, 50, and 100 μg/mL A composition derived from enriched for one or more phenylpropanoid acids and benzoxazinoids. All samples were dissolved in 1% DMSO. A composition derived from enriched for one or more phenylpropanoid acids and benzoxazinoids had a dose-responsive curve with an IC₅₀ of 229 μg/mL, an inhibition constant (Ki) of 119 μg/mL, and a Hill coefficient of 0.80. 6-MBOA did not inhibit 2-lodomelatonin binding to MT1 at the concentrations tested. The IC₅₀ of A composition derived from enriched for one or more phenylpropanoid acids and benzoxazinoids was higher than the highest 6-MBOA tested, but notably, A composition derived from enriched for one or more phenylpropanoid acids and benzoxazinoids inhibited 2-lodomelatonin binding to MT1 at 50 and 100 μg/mL, two concentrations that corresponded to 0.815 μM and 1.63 μM 6-MBOA. This indicated that there were components of A composition derived from enriched for one or more phenylpropanoid acids and benzoxazinoids other than 6-MBOA that competitively bound to the MT1 receptor.

TABLE 11
10 concentrations each of A composition derived from enriched
for one or more phenylpropanoid acids and benzoxazinoids
and 6-MBOA were tested in duplicate for displacement
of [125I]2-lodomelatonin from the MT1 receptor.

| Compound | Concentration | Inhibition | IC₅₀ | Kᵢ | |
|---|---|---|---|---|---|
| Name | (μg/mL) | (%) | (μg/mL) | (μg/mL) | n_{H} |
| A composition | 1600 | 97 | 229 | 119 | 0.80 |
| derived from | 800 | 83 | | | |
| enriched for one | 400 | 65 | | | |
| or more | 200 | 44 | | | |
| phenylpropanoid | 100 | 31 | | | |
| acids and | 50 | 24 | | | |
| benzoxazinoids | 25 | 16 | | | |
| | 12.5 | 11 | | | |
| | 6.25 | 5 | | | |
| | 3.12 | 7 | | | |
| | 1.56 | −1 | | | |
| | 0.78 | 1 | | | |
| Compound | Concentration | Inhibition | IC₅₀ | Kᵢ | |
| Name | (μM) | (%) | (μM) | (μM) | n_{H} |
| 6-MBOA | 1.63 | 8 | (N.C.) | (N.C.) | (N.C.) |
| | 0.815 | 7 | | | |
| | 0.407 | 6 | | | |
| | 0.204 | 7 | | | |
| | 0.102 | 1 | | | |
| | 0.051 | 5 | | | |
| | 0.025 | 11 | | | |
| | 0.013 | 8 | | | |
| | 0.0064 | 3 | | | |
| | 0.0032 | 2 | | | |

N.C. = not calculated

Similarly, for MT2 binding, A composition derived from enriched for one or more phenylpropanoid acids and benzoxazinoids (UP165, Lot #FP041019-01) was tested in duplicate at 10 concentrations (3.12, 6.25, 12.5, 25, 50, 100, 200, 400, 800, and 1600 μg/mL), and 6-MBOA was tested in duplicate at 5 concentrations, (0.815 μM, 1.63 μM, 3.26 μM 6.52 μM, and 13.04 μM), which were equivalent to the 6-MBOA content present in 50, 100, 200, 400, and 800 μg/mL A composition derived from enriched for one or more phenylpropanoid acids and benzoxazinoids. All samples were dissolved in 1% DMSO. A composition derived from enriched for one or more phenylpropanoid acids and benzoxazinoids had a dose-responsive curve with an IC₅₀ of 56.6 μg/mL, an inhibition constant (Kᵢ) of 28.3 μg/mL, and a Hill coefficient of 0.82. 6-MBOA did not inhibit 2-lodomelatonin binding to MT2 at the concentrations tested. Notably, with A composition derived from enriched for one or more phenylpropanoid acids and benzoxazinoids having an IC₅₀ of 56.6 μg/mL, if 6-MBOA was the major contributor to that activity, we would have seen binding activity at all concentrations of 6-MBOA tested. Instead, we didn't see activity at any concentration of 6-MBOA, even at concentrations equivalent to the 6-MBOA in 800 μg/mL A composition derived from enriched for one or more phenylpropanoid acids and benzoxazinoids. This indicated that there were components of A composition derived from enriched for one or more phenylpropanoid acids and benzoxazinoids other than 6-MBOA that competitively bound to the MT2 receptor.

TABLE 12
10 concentrations each of A composition derived from enriched
for one or more phenylpropanoid acids and benzoxazinoids
and 6-MBOA were tested in duplicate for displacement
of [125I]2-lodomelatonin from the MT2 receptor.

| Compound | Concentration | Inhibition | IC₅₀ | Kᵢ | |
|---|---|---|---|---|---|
| Name | (μg/mL) | (%) | (μg/mL) | (μg/mL) | n_{H} |
| A composition | 1600 | 94 | 56.6 | 28.3 | 0.82 |
| derived from | 800 | 85 | | | |
| enriched for one | 400 | 86 | | | |
| or more | 200 | 74 | | | |
| phenylpropanoid | 100 | 59 | | | |
| acids and | 50 | 44 | | | |
| benzoxazinoids | 25 | 38 | | | |
| | 12.5 | 3 | | | |
| | 6.25 | 2 | | | |
| | 3.12 | −13 | | | |
| Compound | Concentration | Inhibition | IC₅₀ | Kᵢ | |
| Name | (μM) | (%) | (μM) | (μM) | n_{H} |
| 6-MBOA | 13.04 | 15 | (N.C.) | (N.C.) | (N.C.) |
| | 6.52 | 11 | | | |
| | 3.26 | 8 | | | |
| | 1.63 | −4 | | | |
| | 0.815 | −12 | | | |

### Example 8: Effect of a Composition Derived from Enriched for One or More Phenylpropanoid Acids and Benzoxazinoids on Sleep Time

Male BALB/C mice (18 g˜22 g) were randomly assigned to groups of vehicle control (sterile water); positive control group, melatonin 1 mg/kg BW (0.1 g/L); low dose group, a composition derived from enriched for one or more phenylpropanoid acids and benzoxazinoids 250 mg/kg BW (25 g/L in sterile water); middle dose group, a composition derived from enriched for one or more phenylpropanoid acids and benzoxazinoids 500 mg/kg BW (50 g/L in sterile water); and high dose group, a composition derived from enriched for one or more phenylpropanoid acids and benzoxazinoids 1000 mg/kg BW (100 g/L in sterile water). Each animal received the respective dose daily for 32 consecutive days. 15 min after the last dose, each group of mice was intraperitoneally injected with 36 mg/kg pentobarbital sodium (in 0.1 mL/10 g). The sleep time of each mouse was recorded and the difference of sleep time between a composition derived from enriched for one or more phenylpropanoid acids and benzoxazinoids-treated groups and vehicle control was determined. As seen in Table 13, mice treated with a composition derived from enriched for one or more phenylpropanoid acids and benzoxazinoids for 32 days showed longer sleep time than that of vehicle control for all the dosages. A similar observation was observed for the positive control, melatonin. Mice treated with the high dose of UP165 showed 11.6±0.2 min (30.6±9.4 vs. 42.2±9.2, P=0.008) increase in sleep time compared to the vehicle control treated animals. Similarly, 10.2±2.4 (P=0.022 vs. vehicle control) and 10.5±0.9 (P=0.017 vs. vehicle control) minutes increases in sleep time were observed for the 250 mg/kg and 500 mg/kg UP165, respectively. As expected, the reference compound, melatonin, showed 12.0±1.3 (P=0.003 vs. vehicle control) minutes increase of sleep time compared to the vehicle control treated mice. UP165 orally administered at a dose level as low as 250 mg/kg resulted in statistically significant prolongation of sleep time in pentobarbital-induced mouse sleep model.

TABLE 13
Effect of a composition derived from enriched for
one or more phenylpropanoid acids and benzoxazinoids
on sleep time induced by pentobarbital sodium

| | Dose | | Sleep Time | |
|---|---|---|---|---|
| Groups | (mg/kg) | Numbers | (min) | P Value |
| Vehicle control | 0 | 15 | 30.6 ± 9.4 | — |
| A composition | 250 | 15 | 40.8 ± 11.8* | 0.022 |
| derived from | 500 | 15 | 41.1 ± 10.3* | 0.017 |
| enriched for one | 1000 | 15 | 42.2 ± 9.2* | 0.008 |
| or more | | | | |
| phenylpropanoid | | | | |
| acids and | | | | |
| benzoxazinoids | | | | |
| Melatonin | 1 | 15 | 42.6 ± 10.7* | 0.003 |

*P-values were compared with vehicle control. Data are expressed as Mean ± SD

### Example 9: Effect of a Composition Derived from Enriched for One or More Phenylpropanoid Acids and Benzoxazinoids on Sleep Latency

Male BALB/C mice (18 g˜22 g) were randomly assigned to groups of vehicle control (sterile water); positive control group, melatonin 1 mg/kg BW (0.1 g/L); low dose group, a composition derived from enriched for one or more phenylpropanoid acids and benzoxazinoids 250 mg/kg BW (25 g/L in sterile water); middle dose group, a composition derived from enriched for one or more phenylpropanoid acids and benzoxazinoids 500 mg/kg BW (50 g/L in sterile water); and high dose group, a composition derived from enriched for one or more phenylpropanoid acids and benzoxazinoids 1000 mg/kg BW (100 g/L in sterile water). Each animal received the respective dose daily for 32 consecutive days. Mice from each group were intraperitoneally injected with 26 mg/kg pentobarbital sodium (0.1 mL/10 g BW), 15 min after the last dose to induce subthreshold hypnosis. The numbers of mice that lose the reflex to turnover to the right side over 1 min after injection of pentobarbital sodium were recorded for 30 min. The incidence of sleeping mice was analyzed to compare the difference between the tested substance groups and vehicle control.

Latency of sleep is the number of animals with loss of the righting reflex in duration of time elapsed after pentobarbital administrations. The hypnotic subthreshold of pentobarbital sodium was determined and found to be 26 mg/kg, where 80%-90% of mice failed to show the loss of reflex to turn over to the right side. As a result, the subthreshold dose of pentobarbital was given to each mouse 15 min after the last dose of test materials to assess the effect of materials on sleep latency. Thirty minutes post pentobarbital injection, the number of mice with no reflex to turn over to the right side over the duration of 1 min was recorded. Data were reported as incidence of sleeping. As depicted Table 14, 10 out of 15 mice (67%) for the high dose of UP165 and 12 out of 15 mice (80%) for the melatonin group were found to have shortened latency. These incidences were statistically significant for both the high dose of UP165 and melatonin compared to the vehicle control animals. In comparison, the incidence in the vehicle-treated group was only 20%. Positive trends (60% of mice both in the 250 and 500 mg/kg group) were also observed for the low and mid doses of UP165. It was clearly evident that mice treated with high dose of a composition derived from enriched for one or more phenylpropanoid acids and benzoxazinoids showed statistically significant improved sleeping incidence when compared to vehicle control. This incidence was almost comparable to the one observed for the positive control melatonin.

TABLE 14
Effect of a composition derived from enriched for one or
more phenylpropanoid acids and benzoxazinoids on the latent
time of sleeping induced by pentobarbital sodium

| | Dose | Total | Sleeping | | |
|---|---|---|---|---|---|
| Groups | (mg/kg) | No. | No. | Incidence | P value |
| Vehicle control | 0 | 15 | 3 | 20% | — |
| A composition | 250 | 15 | 9 | 60% | 0.060 |
| derived from | 500 | 15 | 9 | 60% | 0.060 |
| enriched for one | 1000 | 15 | 10 | 67%* | 0.025 |
| or more | | | | | |
| phenylpropanoid | | | | | |
| acids and | | | | | |
| benzoxazinoids | | | | | |
| Melatonin | 1 | 15 | 12 | 80%* | 0.003 |

*P-values were compared with vehicle control. Data are expressed as Mean ± SD

### Example 10: Acute Effect of a Composition Derived from Enriched for One or More Phenylpropanoid Acids and Benzoxazinoids on Sleep

Male BALB/C mice (18 g˜22 g) were randomly assigned to groups of vehicle control (sterile water); positive control group, melatonin 1 mg/kg BW (0.1 g/L); low dose group, a composition derived from enriched for one or more phenylpropanoid acids and benzoxazinoids 250 mg/kg BW (25 g/L in sterile water); middle dose group, a composition derived from enriched for one or more phenylpropanoid acids and benzoxazinoids 500 mg/kg BW (50 g/L in sterile water); and high dose group, a composition derived from enriched for one or more phenylpropanoid acids and benzoxazinoids 1000 mg/kgBW (100 g/L in sterile water). Mice were administered the respective treatment group and observed for 60 min monitor the direct impact of test compounds on sleep following a single oral administration.

When naive mice are placed into a supine position, they instantaneously turn to the upright position. However, mice under the hypnotic dose of pentobarbital remain on the supine position for measurable amount of time. Sleeping was indexed as the disappearance of reflex to turnover to the right side, and mouse was considered asleep when the time to turn over to the side was over 30 s after mouse was treated with a composition derived from enriched for one or more phenylpropanoid acids and benzoxazinoids. In the current study, none of the treatment groups induce sleep following a single oral administration (Table 15).

TABLE 15
Effect of a composition derived from enriched for
one or more phenylpropanoid acids and benzoxazinoids
on sleep following a single oral administration

| | Dose | Total | Sleeping | | |
|---|---|---|---|---|---|
| Groups | (mg/kg) | No. | No. | Incidence | P value |
| Vehicle control | 0 | 15 | 0 | 0% | NA |
| A composition | 250 | 15 | 0 | 0% | NA |
| derived from | 500 | 15 | 0 | 0% | NA |
| enriched for one | 1000 | 15 | 0 | 0% | NA |
| or more | | | | | |
| phenylpropanoid | | | | | |
| acids and | | | | | |
| benzoxazinoids | | | | | |
| Melatonin | 1 | 15 | 0 | 0% | NA |

*P-values were compared with vehicle control.

### Example 11: A Pilot Proof of Concept Study to Evaluate a Composition Derived from Enriched for One or More Phenylpropanoid Acids and Benzoxazinoids (UP165) on Sleep Time, and Sleep Quality in Normal Healthy Adults

The effect on sleep was evaluated in double blind placebo-controlled clinical study. This study enrolled 45 volunteers to participate in a 6-week trial of the effects of a composition derived from enriched for one or more phenylpropanoid acids and benzoxazinoids (UP165, immature corn leaf extract standardized at not less than 0.2% 6-MBOA) on sleep quality.

Subjects were instructed to consume the supplement or placebo for 4-weeks after a 2-week baseline period (no-supplementation). The subjects were instructed to take the supplement materials orally approximately 60 minutes before bedtime daily for 4 weeks. Forty-two subjects completed the 4-week supplementation period. Three subjects were lost to follow-up (not to adverse effects)—1 from the 250 mg group and 2 from the 500 mg group.

The analysis compares the average of the two baseline weeks to the average of the 4 weeks of supplementation and changes observed from average baseline values to week 1, week 2, week 3 and week 4 values.

TABLE 16
The subject population at the end of the study

| Age range 19-73 years old | Average age = 43 years old |
|---|---|
| Female | 23 |
| Male | 19 |
| Total | 42 |

TABLE 17
The study subjects were divided into 3 supplementation Groups.

| Study Groups | N |
|---|---|
| A: 250 mgs of UP165 | 14 |
| B: 500 mgs of UP165 | 13 |
| C: Placebo Group | 15 |

Data were collected on the following parameter, twice during baseline (prior to supplementation) and weekly for 4 additional weeks during the 6-week study.
Sleep Quality was measured using the Garmin Viviosmart® 4 fitness activity tracker. The tracker includes advanced sleep monitoring with REM sleep, light sleep, deep sleep, and movement throughout the night.
Sleep Quality was also measured using the validated Pittsburgh Sleep Quality Index; (PSQI). The PSQI was designed to evaluate overall sleep quality. Each of the questionnaire's 19 self-reported items belongs to one of seven subcategories: subjective sleep quality, sleep latency, sleep duration, habitual sleep efficiency, sleep disturbances, use of sleeping medication, and daytime dysfunction.
Salivary Cortisol Level: Cortisol is a major glucocorticosteroid hormone produced by the adrenal cortex, it is actively involved in multiple biological pathways, salivary cortisol is unbound to protein and there is a high correlation between salivary cortisol and serum cortisol.

### Example 12: Up165 Supplementation Improved the State of Deep Sleep and Hence Quality of Sleep in Dose Correlated Manner

The basic structural organization of normal sleep involves the REM and non-rem sleep stages. A sleep episode begins with a short period of Non-REM stage 1 progressing through stage 2, followed by stages 3 and 4 and finally to REM. Individuals do not remain in one stage, but, rather, revolve between stages of Non-REM and REM throughout the night. While the non-REM sleep constitutes about 75-80% of total time spent in sleep, the REM sleep constitutes the remaining 20-25%. As such, those supplements with the effect in influencing the non-rem stage of sleep will have a potential in impacting the larger portion of the night. If the impact is on the positive direction (i.e. improving deep sleep) as observed for UP165, it will lead to an improved quality of sleep. Sleep, particularly the slow wave deep sleep, inhibits cortisol secretion and the rise of cortisol secretion during sleep could lead to awakenings.

As seen in Table 18 below, there was a progressive increase in the state of deep sleep for subjects who were supplanted with either doses of UP165. The time spent in the deep sleep stage was statistically significant for subjects who were given UP165 at 500 mg/day as early as the second week of supplementation and remained significant for the duration of study except at week 3 which showed strong trend. The increase of deep sleep was statistically significant on week 4 for the 250 mg/day group though marked increases in deep sleep timing was observed as of week 1. When the percent of increase from the base line was computed (Table 19), it was found that UP165 at 500 mg/kg produced 27.9-47.4% and the 250 mg/kg caused 30.5-45.6% increase in deep sleep time. When the 4 weeks average of deep sleep increase was summed up, 38.8% and 31.4% increase in deep sleep stages were observed for the 250 mg/day and 500 mg/day UP165, respectively. In contrast, subjects in the placebo group showed decrease in the deep sleep stage in weeks 2- and 3. When the overall changes in the deep sleep stage was calculated, it was found to be −3% from the baseline for the Placebo group. These objective measures clearly showed that UP165 is a dietary supplement with marked impact on sleep quality as reflected by statistically significant increased deep sleep time. When the prolonged deep sleep time was compared against the placebo group, subjects in the UP165 group showed statistically significant increase in deep sleep stage as early as week 2 and remained significant for the course of the study except on week 3 when both dosages showed marked increase with P=0.0551 and P=0.0742, respectively, for the 250 mg/day and 500 mg/day (Table 20).

TABLE 18
Effect of UP 165 on the average Deep sleep values on healthy subjects

| | | Deep Sleep (Mean ± SD) | | | | | |
|---|---|---|---|---|---|---|---|
| | | | | | | | Average |
| Group | N | Baseline | Week-1 | Week-2 | Week-3 | Week-4 | (Wk1-4) |
| UP165 | 12 | 3825.9 ± | 5571.9 ± | 5154.5 ± | 4992.75 ± | 5518.5 ± | 5309.42 ± |
| (250 mg/day) | | 1653.3 | 3340.1 | 2457.5 | 2184.8 | 2259.8 | 2185.4 |
| | | | (p = 0.099) | (p = 0.073) | (p = 0.089) | (p = 0.051) | (p = 0.045) |
| UP165 | 13 | 4055.5 ± | 4506.5 ± | 4916.2 ± | 5125.1 ± | 5665.3 ± | 5053.3 ± |
| (500 mg/day) | | 2058.8 | 2083.4 | 2067.4 | 2965.9 | 3099.3 | 2368.0 |
| | | | (p = 0.267) | (p = 0.025) | (p = 0.096) | (p = 0.014) | (p = 0.022) |
| Placebo | 14 | 3473.9 ± | 4019.0 ± | 3002.6 ± | 2710.9 ± | 3740.9 ± | 3368.4 ± |
| | | 1644.1 | 1358.3 | 1460.8 | 996.9 | 2226.3 | 884.0 |
| | | | (p = 0.289) | (p = 0.157) | (p = 0.161) | (p = 0.719) | (p = 0.797) |

Data in parenthesis are p-vales against baseline for each treatment group

TABLE 19
Percent baseline change of UP165 deep
sleep effect on healthy subjects

| | | Deep Sleep (Percent difference of baseline) | | | | |
|---|---|---|---|---|---|---|
| | | | | | | Average |
| Group | N | Week-1 | Week-2 | Week-3 | Week-4 | (Wk1-4) |
| UP165 | 12 | 45.6% | 34.7% | 30.5% | 44.2% | 38.8% |
| (250 mg/day) | | | | | | |
| UP165 | 13 | 17.2% | 27.9% | 33.3% | 47.4% | 31.4% |
| (500 mg/day) | | | | | | |
| Placebo | 14 | 15.7% | −13.6% | −22.0% | 7.7% | −3.0% |

TABLE 20
Statistical significance of UP165
on deep sleep compared to placebo

| | | Deep Sleep (Paired t-test vs placebo p-value) | | | | |
|---|---|---|---|---|---|---|
| | | | | | | Average |
| Group | N | Week-1 | Week-2 | Week-3 | Week-4 | (Wk1-4) |
| UP165 | 12 | 0.1234 | 0.0109 | 0.0018 | 0.0551 | 0.0055 |
| (250 mg/day) | | | | | | |
| UP165 | 13 | 0.4748 | 0.0098 | 0.0081 | 0.0742 | 0.0200 |
| (500 mg/day) | | | | | | |

### Example 13: UP165 Supplementation Caused Statistically Significant Reductions in Cortisol Level

Cortisol, one of the major glucocorticoid hormones secreted by the adrenal cortex, is amongst the hormones that regulate human homeostasis. Increased plasma cortisol level is associated with impaired HPA feedback regulation that will lead to sleep fragmentation and poor quality. Poor sleep quality and hypercortisolemia are the most often reported changes in healthy subjects with sleep disturbance because of hyperactive HPA axis. Decreased cortisol level and hence normalized APA axis feedback response lead to adequate sleep and improved sleep quality. In this clinical study, subjects supplemented with UP165 showed progressive decrease in salivary cortisol level through the course of study (Table 21). A marked decrease in cortisol level was observed for the 500 mg/day group of subjects at week 3 (P=0.06 verses baseline) followed by statistically significant decrease on week 4. Subjects in the 250 mg/day group showed statistically significant reduction in the cortisol level on week 4.

Interestingly as shown in the Table 22, while the 250 mg/day and 500 mg/day UP165 supplemented subjects experienced an 11% and 15% reduction in cortisol level in week-1, respectively, in the same time frame, the placebo group showed a 6.2% increase in cortisol level. These patterns of cortisol changes were sustained for the duration of study and by the end of the study period when the 4 weeks values were averaged, 20.2%, and 24.7% decrease in the cortisol levels were observed for the 250 mg/day and 500 mg/day UP165, respectively. In contrast, the Placebo received subjects showed a 5.3% increase in cortisol level for the 4-week average. These reductions in cortisol level were dose correlated for the UP165 group. Reductions of cortisol level such as 11.0, 21.8, 20.2 and 27.5% for the 250 mg/day and 15.0, 18.2, 29.5 and 36.3% for the 500 mg/day UP165 were observed for weeks 1-4, respectively.

TABLE 21
Effect of UP165 on the average salivary cortisol level

| | | Cortisol (Mean ± SD) | | | | | |
|---|---|---|---|---|---|---|---|
| | | | | | | | Average |
| Group | N | Baseline | Week-1 | Week-2 | Week-3 | Week-4 | (Wk1-4) |
| UP165 | 12 | 3529 ± 1686 | 3139 ± 1670 | 2759 ± 940 | 2815 ± 1838 | 2557 ± 972 | 2818 ± 1064 |
| (250 mg/day) | | | (p = 0.418) | (p = 0.161) | (p = 0.261) | (p = 0.040) | (0.133) |
| UP165 | 13 | 2920 ± 1008 | 2482 ± 1189 | 2389 ± 1263 | 2059 ± 1067 | 1861 ± 749 | 2198 ± 889 |
| (500 mg/day) | | | (p = 0.180) | (p = 0.163) | (p = 0.060) | (p = 0.008) | (p = 0.039) |
| Placebo | 14 | 3294 ± 1092 | 3498 ± 2916 | 3616 ± 2063 | 3351 ± 2288 | 3406 ± 2365 | 3468 ± 1638 |
| | | | (p = 0.709) | (p = 0.587) | (p = 0.921) | (p = 0.864) | (p = 0.693) |

Data in parenthesis are p-vales against baseline for each treatment group

TABLE 22
Percent baseline change of UP165 on salivary
cortisol level on healthy subjects

| | | Cortisol (Percent difference of baseline) | | | | |
|---|---|---|---|---|---|---|
| | | | | | | Average |
| Group | N | Week-1 | Week-2 | Week-3 | Week-4 | (Wk1-4) |
| UP165 | 12 | −11.0% | −21.8% | −20.2% | −27.5% | −20.2% |
| (250 mg/day) | | | | | | |
| UP165 | 13 | −15.0% | −18.2% | −29.5% | −36.3% | −24.7% |
| (500 mg/day) | | | | | | |
| Placebo | 14 | 6.2% | 9.8% | 1.7% | 3.4% | 5.3% |

When weekly values were compared for each treatment group relative to placebo (Table 23), the 500 mg/day group showed statistically significant reduction in cortisol level on week 4 with marked differences in week 2 and 3. These reduction in cortisol level was also statistically significant for the 500 mg/day group when the 4 week average value was compared against the placebo group. These cortisol findings align well with the deep sleep findings described above affirming the improved sleep quality and efficiency experienced as a result of UP165 supplementations.

TABLE 23
Statistical significance of UP165 on salivary
cortisol level compared to placebo

| | | Cortisol (Paired t-test vs placebo, p-value) | | | | |
|---|---|---|---|---|---|---|
| | | | | | | Average |
| Group | N | Week-1 | Week-2 | Week-3 | Week-4 | (Wk1-4) |
| UP165 | 12 | 0.6123 | 0.1688 | 0.5012 | 0.2257 | 0.2243 |
| (250 mg/day) | | | | | | |
| UP165 | 13 | 0.1270 | 0.0767 | 0.0755 | 0.0336 | 0.0205 |
| (500 mg/day) | | | | | | |

### Example 14: UP165 Supplementation Resulted in Increase in Rem Stage of Sleep

Among the stages of sleep, REM sleep is defined by the presence of desynchronized brain wave activity, muscle atonia, and bursts of rapid eye movements. During the initial cycle, the REM period may last only 1 to 5 minutes; however, it becomes progressively prolonged as the sleep episode progresses to cover 20-25 percent of the night. Dreaming is most often associated with REM sleep. In the current clinical study, subjects who were supplemented with either doses of UP165 showed marked increase in the REM stage of sleep. As shown in the Table 25, there were 19.6-22.5% for the 250 mg/day and 15.3-23.2% for the 500 mg/day UP165 increase in the REM stage of sleep during the 4 weeks of study. It is worth noting that, in week 1, while the 250 mg/day and 500 mg/day UP165 group showed 22.5% and 23.2% increase in the rem stage of sleep, respectively, the placebo group showed a 2.48% decrease in the rem sleep stage. After the end of the 4 weeks study period, when the 4 weeks values were averaged, there were 21.4%, 19.8% and 1.55% increase in the REM stages of sleep for the 250 mg/day UP165, 500 mg/day UP165 and placebo, respectively.

TABLE 24
Effect of UP 165 on the average REM values on healthy subjects

| | | REM (Mean ± SD) | | | | | |
|---|---|---|---|---|---|---|---|
| | | | | | | | Average |
| Group | N | Baseline | Week-1 | Week-2 | Week-3 | Week-4 | (Wk1-4) |
| UP165 | 12 | 5293 ± 2443 | 6483 ± 3193 | 6430 ± 2658 | 6331 ± 2886 | 6470 ± 2688 | 6429 ± 2640 |
| (250 mg/day) | | | (p = 0.269) | (p = 0.266) | (p = 0.315) | (p = 0.160) | (p = 0.228) |
| UP165 | 13 | 5789 ± 1920 | 7132 ± 2276 | 7105 ± 1741 | 6673 ± 1084 | 6827 ± 1998 | 6934± 1529 |
| (500 mg/day) | | | (p = 0.169) | (p = 0.091) | (p = 0.204) | (p = 0.260) | (p = 0.152) |
| Placebo | 14 | 7593 ± 2615 | 7404 ± 1988 | 8126 ± 3164 | 8007 ± 3501 | 7304 ± 3305 | 7710 ± 2724 |
| | | | (p = 0.666) | (p = 0.146) | (p = 0.469) | (p = 0.666) | (p = 0.816) |

Data in parenthesis are p-vales against baseline for each treatment group

TABLE 25
Percent baseline change of UP165 on
REM sleep stage on healthy subjects

| | | REM (Percent difference of baseline) | | | | |
|---|---|---|---|---|---|---|
| | | | | | | Average |
| Group | N | Week-1 | Week-2 | Week-3 | Week-4 | (Wk1-4) |
| UP165 | 12 | 22.5% | 21.5% | 19.6% | 22.2% | 21.4% |
| (250 mg/day) | | | | | | |
| UP165 | 13 | 23.2% | 22.7% | 15.3% | 17.9% | 19.8% |
| (500 mg/day) | | | | | | |
| Placebo | 14 | −2.48% | 7.02% | 5.45% | −3.81% | 1.55% |

### Example 15: Up165 Supplementation Showed Minimal Impact on the Light Stage of Sleep

Light sleep stages constitute stages 1 and 2. Stage 1 sleep serves as a transitional role in sleep-stage cycling. The average individual's sleep episode begins in stage 1. This stage usually lasts 1 to 7 minutes in the initial cycle, constituting 2 to 5 percent of total sleep and followed by a stage 2 sleep. Stage 2 sleep lasts approximately 10 to 25 minutes in the initial cycle and lengthens with each successive cycle. While an individual in stage 1 sleep is easily interrupted by a disruptive noise an individual in stage 2 sleep requires more intense stimuli than in stage 1 to be awaken. As depicted in the table 26 and 27 below, UP165 supplementation has minimal effect on the light sleep stage of an individual. The treatment groups performed as equal as the placebo group in the light sleep stage indicating subjects followed a normal habitual sleep patterns in response to light-dark cycles. These stages of sleep are known to be regulated by the circadian rhythmicity for a normal sleep and awake cycling than a physiologic homeostasis modulated by cortisol level.

TABLE 26
Effect of UP165 on light sleep stage

| | | Light sleep (Mean ± SD) | | | | | |
|---|---|---|---|---|---|---|---|
| | | | | | | | Average |
| Group | N | Baseline | Week-1 | Week-2 | Week-3 | Week-4 | (Wk1-4) |
| UP165 | 12 | 16519 ± 1673 | 15682 ± 2842 | 16102 ± 2909 | 15655 ± 2164 | 16330 ± 2170 | 15942 ± 2083 |
| (250 mg/day) | | | (p = 0.243) | (p = 0.575) | (p = 0.114) | (p = 0.812) | (p = 0.286) |
| UP165 | 13 | 17944 ± 2457 | 17661 ± 3391 | 18052 ± 2040 | 18793 ± 2814 | 17873 ± 2676 | 18095 ± 2261 |
| (500 mg/day) | | | (p = 0.644) | (p = 0.828) | (p = 0.345) | (p = 0.929) | (p = 0.783) |
| Placebo | 14 | 17913 ± 3037 | 17416 ± 3231 | 17690 ± 3373 | 17274 ± 3261 | 18276 ± 3997 | 17664 ± 3018 |
| | | | (p = 0.613) | (p = 0.858) | (p = 0.333) | (p = 0.390) | (p = 0.731) |

Data in parenthesis are p-vales against baseline for each treatment group

TABLE 27
Percent baseline change effect of UP165 on healthy subjects

| | | Light Sleep (Percent difference of baseline) | | | | |
|---|---|---|---|---|---|---|
| | | | | | | Average |
| Group | N | Week-1 | Week-2 | Week-3 | Week-4 | (Wk1-4) |
| UP165 | 12 | −5.1% | −2.5% | −5.2% | −1.1% | −3.5% |
| (250 mg/day) | | | | | | |
| UP165 | 13 | −1.6% | 0.6% | 4.7% | −0.4% | 0.8% |
| (500 mg/day) | | | | | | |
| Placebo | 14 | −2.8% | −1.2% | −3.6% | 2.0% | −1.4% |

### Example 16: UP165 Supplementation Reduced the State of Awakening at Early Periods of Study

In some context, state of awakening is considered as the last stage of sleep. In the current clinical study, supplementation of UP165 has statistically significant reduction in the state of awakening at the early phase of the study period (i.e. Table 28, week 1). Subjects in the 500 mg/day group showed a 28.8% reduction in the state of awakening when compared to the baseline. In the same time period, (following a week of supplementation), subjects who received the placebo showed a 21.3% increase in the state of awakening relative to their baseline values (Table 29). When the 4 weeks values were averaged, it was found that the UP165 supplementation showed 6.4% and 6.1% reductions in the state of awakening for the 250 mg/day and 500 mg/day, respectively. In contrast, when the 4 weeks values were averaged, the placebo group showed a 12.3% increase in the state of awakening compared to baseline. These findings indicate that supplementation of UP165 to healthy subjects will produce a better sleep quality by minimizing the state of being awake.

TABLE 28
Effect of UP 165 on awake time

| | | Awake time (Mean ± SD) | | | | | |
|---|---|---|---|---|---|---|---|
| | | | | | | | Average |
| Group | N | Baseline | Week-1 | Week-2 | Week-3 | Week-4 | (Wk1-4) |
| UP165 | 12 | 681 ± 679 | 661 ± 743 | 659 ± 556 | 543 ± 668 | 688 ± 733 | 638 ± 620 |
| (250 mg/day) | | | (p = 0.980) | (p = 1.00) | (p = 0.393) | (p = 0.655) | (p = 0.685) |
| UP165 | 13 | 519 ± 219 | 369 ± 132 | 523 ± 288 | 602 ± 260 | 456 ± 276 | 487 ± 156 |
| (500 mg/day) | | | (p = 0.049) | (p = 0.962) | (p = 0.440) | (p = 0.602) | (p = 0.692) |
| Placebo | 14 | 391 ± 264 | 475 ± 343 | 442 ± 295 | 455 ± 301 | 388 ± 204 | 440 ± 240 |
| | | | (p = 0.183) | (p = 0.336) | (p = 0.232) | (p = 0.885) | (p = 0.260) |

Data in parenthesis are p-vales against baseline for each treatment group

TABLE 29
Percent baseline change effect of UP165
supplementation on healthy subjects

| | | Awake time (Percent difference of baseline) | | | | |
|---|---|---|---|---|---|---|
| | | | | | | Average |
| Group | N | Week-1 | Week-2 | Week-3 | Week-4 | (Wk1-4) |
| UP165 | 12 | −3.0% | −3.3% | −20.3% | 1.0% | −6.4% |
| (250 mg/day) | | | | | | |
| UP165 | 13 | −28.8% | 0.7% | 16.0% | −12.1% | −6.1% |
| (500 mg/day) | | | | | | |
| Placebo | 14 | 21.3% | 12.8% | 16.2% | −1.0% | 12.3% |

### Example 17: Up165 Supplementation Moderately Increased Total Sleep Time

Compared to the baseline, the composition derived from enriched for one or more phenylpropanoid acids and benzoxazinoids (UP165) resulted in moderate but statistically significant increase in total sleep time when supplemented to healthy subjects at a dosage level of 500 mg/day. At the end of the 4-week study period, there was 8.0% (an average of 32 minutes/night) and 9.1% (an average of 40 minutes/night) prolonged sleep total time for the 250 mg and 500 mg/day UP165 groups, respectively (Table 30). Total sleep time was unaffected for the Placebo group. These data further indicate that the significant impact of UP165 supplementation on healthy subjects is on sleep quality and efficiency than quantity.

TABLE 30
Effect of UP165 supplementation on total sleep time

| | | Total sleep time/Sleep quantity | | | | |
|---|---|---|---|---|---|---|
| | | | | % difference | p-value | p-value |
| Group | N | BL | Wk-4 | of BL | vs BL | vs Placebo |
| UP165 | 12 | 25638 ± 4481 | 2768 ± 3995 | 8.0 | 0.1398 | 0.7730 |
| (250 mg/day) | | | | | | |
| UP165 | 13 | 27578 ± 4077 | 30082 ± 3411 | 9.1 | 0.0327 | 0.2532 |
| (500 mg/day) | | | | | | |
| Placebo | 14 | 28980 ± 2739 | 28742 ± 4810 | −0.8 | 0.4626 | — |

### Example 18: Up165 Supplementation Improved Pittsburgh Sleep Quality Index

The Pittsburgh Sleep Quality Index (PSQI) is a self-rated questionnaire which assesses sleep quality and disturbances over a 4-week time interval. Nineteen individual items generate seven component scores such as subjective sleep quality, sleep latency, sleep duration, habitual sleep efficiency, sleep disturbances, use of sleeping medication, and daytime dysfunction. The sum of scores for these seven components yields one global score where higher scores correspond to worsening of sleep quality and efficiency (Buysse et al., 1989).

In agreement with the objective measures documented in this invention (Table 31), subjects who were supplemented with the composition derived from enriched for one or more phenylpropanoid acids and benzoxazinoids (UP165) showed statistically significant improvement in self-rated questionnaires indicating the effect of UP165 in enhancing sleep quality and efficiency in healthy subjects. UP165 supplementation produced significantly improved sleep quality as early as week 1 and its effect sustained throughout the 4-week duration. During this supplementation period, as shown in Table 32, UP165 caused improvement in sleep quality as high as 48.9% from baseline when it was administered at 500 mg/day and 35.1% when it was given at 250 mg/day to healthy subjects. The placebo effect observed in week 1 for the placebo receiving group was nonexistent for the remainder of the study period. When the 4-week sleep quality improvement was averaged, it was found that subjects who were supplemented with UP165 to have 34.7% and 31.9% increase in sleep quality and efficiency at 250 mg/day and 500 mg/day, respectively. On the other hand, the change for the placebo group was only 3.7%. These PSQI data is a confirmation that UP165 supplementation indeed has significant impact on sleep quality and efficiency improvements. These improvements are a direct reflection of significant reductions in cortisol level and increased deep sleep stage of sleep which were regulated by the HPA axis feedback of the sleep cycle.

TABLE 31
Effect of UP165 supplementation on Pittsburgh Sleep Quality Index

| | | PSQI Global Score (Mean ± SD) | | | | | |
|---|---|---|---|---|---|---|---|
| | | | | | | | Average |
| Group | N | Baseline | Week-1 | Week-2 | Week-3 | Week-4 | (Wk1-4) |
| UP165 | 12 | 6.93 ± 3.79 | 4.50 ± 2.68 | 4.57 ± 2.47 | 4.50 ± 2.24 | 4.50 ± 2.88 | 4.52 ± 2.33 |
| (250 mg/day) | | | (p = 0.003) | (p = 0.006) | (p = 0.003) | (p = 0.005) | (p = 0.002) |
| UP165 | 13 | 7.23 ± 2.59 | 5.77 ± 2.68 | 5.62 ± 3.66 | 3.69 ± 2.32 | 4.62 ± 3.52 | 4.92 ± 2.42 |
| (500 mg/day) | | | (p = 0.013) | (p = 0.026) | (p = 0.002) | (p = 0.009) | (p = 0.001) |
| Placebo | 14 | 5.86 ± 2.71 | 4.00 ± 2.54 | 6.07 ± 3.29 | 6.43 ± 3.80 | 6.07 ± 3.00 | 5.64 ± 1.83 |
| | | | (p = 0.023) | (p = 0.836) | (p = 0.648) | (p = 0.813) | (p = 0.650) |

Data in parenthesis are p-vales against baseline for each treatment group
PSQI—Pittsburgh Sleep Quality Index

TABLE 32
Percent baseline change effect of UP165 supplementation on PSQI

| | | PSQI Global Score (Percent difference of baseline) | | | | |
|---|---|---|---|---|---|---|
| | | | | | | Average |
| Group | N | Week-1 | Week-2 | Week-3 | Week-4 | (Wk1-4) |
| UP165 | 12 | −35.1% | −34.0% | −35.1% | −35.1% | −34.7% |
| (250 mg/day) | | | | | | |
| UP165 | 13 | −20.2% | −22.3% | −48.9% | −36.2% | −31.9% |
| (500 mg/day) | | | | | | |
| Placebo | 14 | −31.6% | 3.6% | 9.7% | 3.6% | −3.7% |

PSQI—Pittsburgh Sleep Quality Index

### Example 19: Supplemental Data Analysis for the Randomized Double-Blind Placebo Controlled Clinical Trial of a Composition Derived from Enriched for One or More Phenylpropanoid Acids and Benzoxazinoids

Study Design:

The study enrolled 45 ((age range 19-73) moderately stressed (clinically undiagnosed), normal, healthy adults (24 female and 21 male subjects) participants. Subjects were randomized to receive the composition derived from enriched for one or more phenylpropanoid acids and Benzoxazinoids™ (supplement at 250 mg/day—Group A and 500 mg/day—Group B) or a non-active corn starch placebo (placebo—Group C). Subjects were instructed to consume the supplement or placebo approximately 60 minutes before bedtime daily for 4-weeks after a 2-week baseline period (no supplementation). Following baseline monitoring, Sleep Quality (Deep/REM time using the Garmin sleep tracker), Profile of Mood States (POMS) and Pittsburgh Sleep Quality Survey were monitored for each subject in each group for a period of 4 weeks. Salivary cortisol level was also determined for all subjects on Day 0 and weekly for four weeks. Supplemental blinded data analysis was performed by Mark Payton, PhD.

Statistical Analysis:

To assess the differences in Groups, analysis of covariance was used with a repeated measures model with Week as the repeated variable and subject (denoted by ID) as the repeated experimental unit. An autoregressive covariance structure with a Kenward-Roger degree of freedom adjustment was used to adjust for variance differences. The base averages were used as covariates. Least square means (adjusted for differences in the covariate) and standard errors were calculated, and simple effect comparisons of group given week and week given group were assessed using a significance level of 0.05.

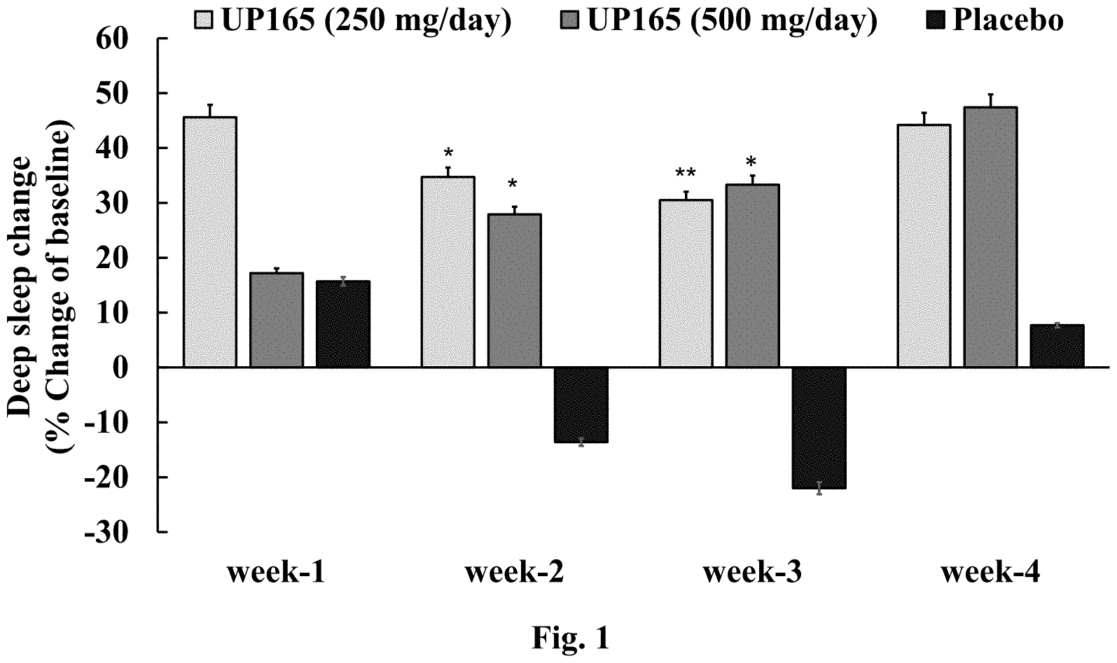

Results Highlights (Shown in FIGS. 1-5)

Deep Sleep (FIG. 1)
Supplementation of a composition derived from enriched for one or more phenylpropanoid acids and benzoxazinoids at 250 mg/day showed statistically significant increase in the deep sleep time compared to placebo on weeks 2 (p=0.0219) and 3 (p=0.0147) with a positive trend on week 4 (p=0.0621).
Supplementation of a composition derived from enriched for one or more phenylpropanoid acids and benzoxazinoids at 500 mg/day showed statistically significant increase in the deep sleep time compared to placebo on week 3 (p=0.0131) with positive trends on week 2 (p=0.057) and week 4 (p=0.0554).
The increase in deep sleep time among all the groups was found statistically significant (p=0.0318).

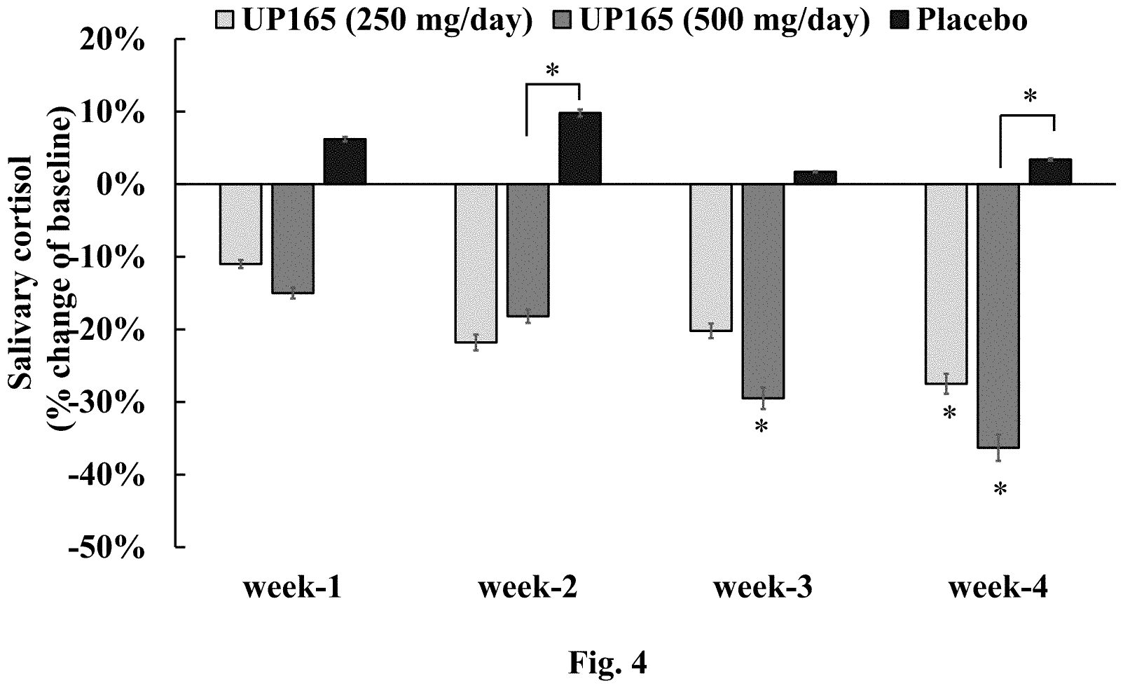

Cortisol (FIG. 4)
Supplementation of a composition derived from enriched for one or more phenylpropanoid acids and benzoxazinoids at 500 mg/day showed statistically significant decrease in the salivary cortisol level compared to placebo on week 4 (p=0.0229) with positive trends on week 2 (p=0.0757) and week 3 (p=0.0604).
The decrease in salivary cortisol level among all the groups was found statistically significant (p=0.0289).

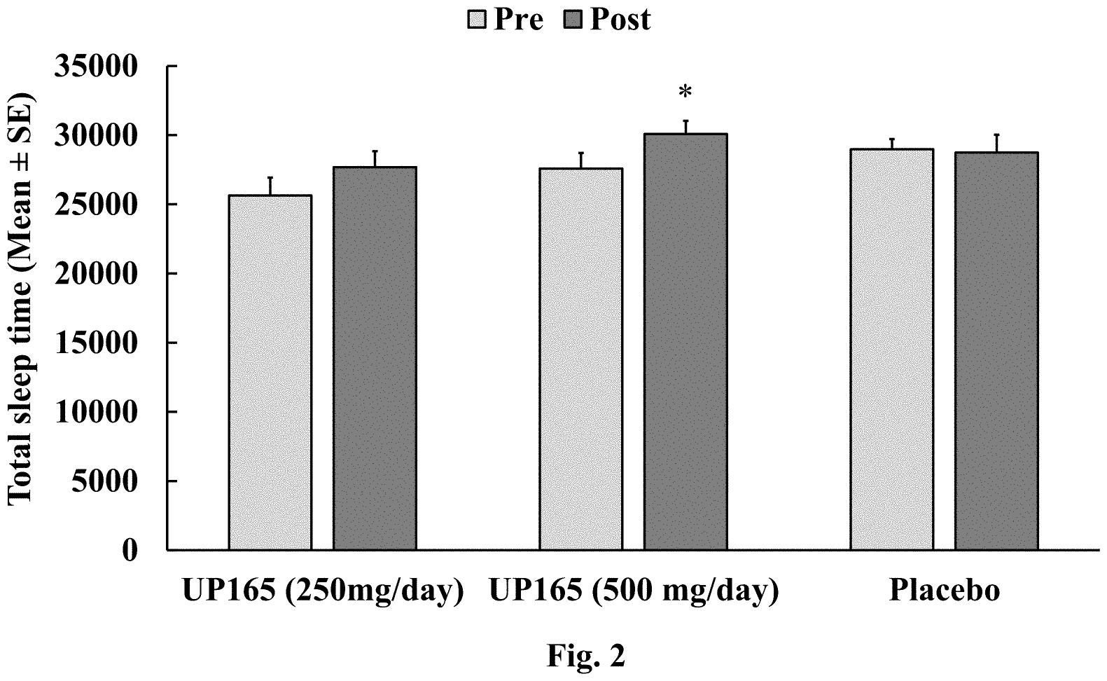

Total Sleep Time (FIG. 2)
Compared to the placebo group, statistically significant increase in the total sleep time was observed for participants supplemented with 500 mg/day a composition derived from enriched for one or more phenylpropanoid acids and benzoxazinoids with p=0.050 across the weeks.

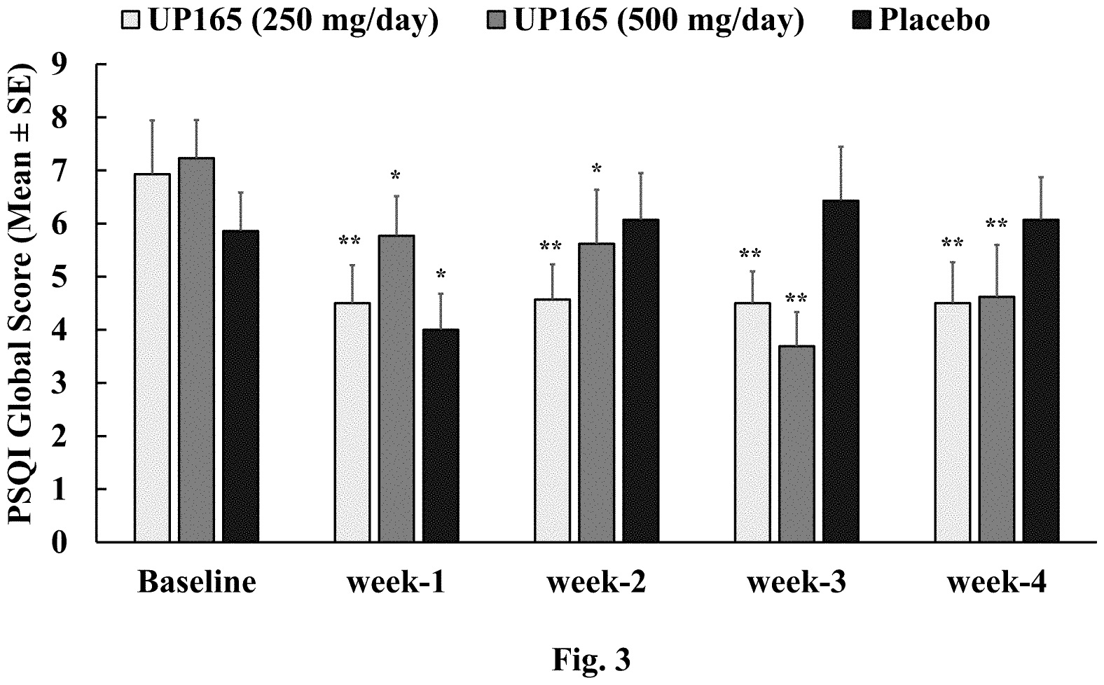

PSQI (FIG. 3)
Supplementation of a composition derived from enriched for one or more phenylpropanoid acids and benzoxazinoids at 250 mg/day showed statistically significant improvement in quality of sleep as measured in PSQI compared to placebo on weeks 3 (p=0.0325) with positive trends on 2 (p=0.0690) and week 4 (p=0.0584).
Supplementation of a composition derived from enriched for one or more phenylpropanoid acids and benzoxazinoids at 500 mg/day showed statistically significant improvement in quality of sleep as measured in PSQI compared to placebo on week 3 (p=0.0022) with a positive trend on week 4 (p=0.0756).
The improvement in quality of sleep as measured by PSQI among the groups was found statistically significant with P=0.0273.

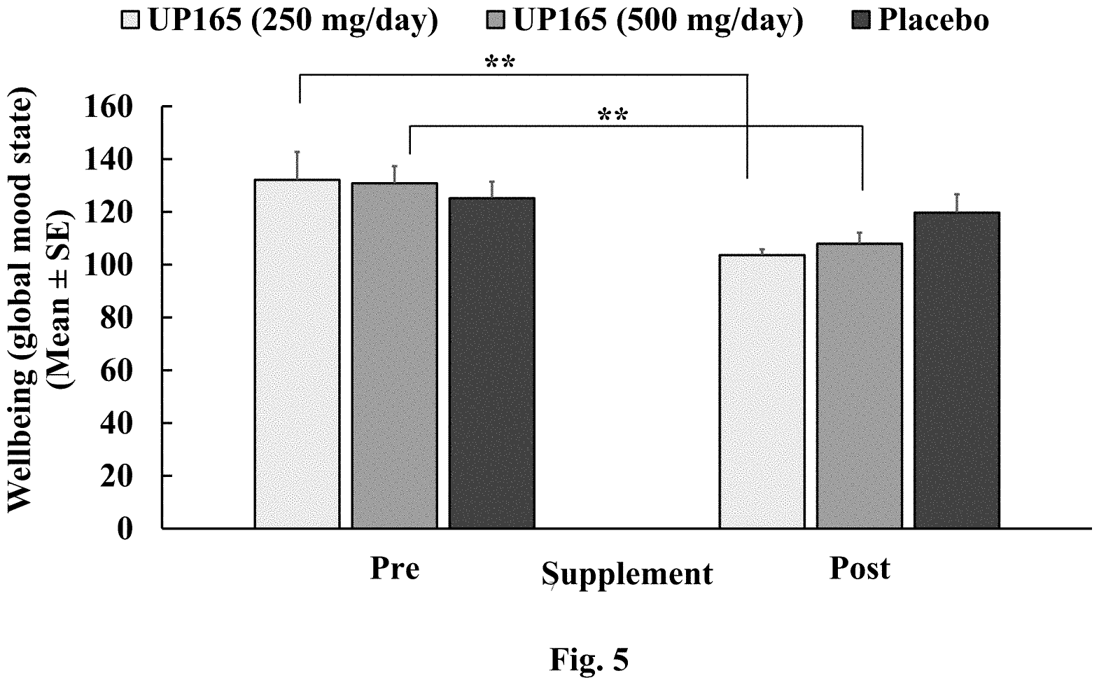

POMS Over all Well being (FIG. 5)
Compared to placebo, participants supplemented with a composition derived from enriched for one or more phenylpropanoid acids and benzoxazinoids at 250 mg/day showed statistically significant increase in the overall wellbeing across the weeks with P=0.0020, 0.0117, 0.0006 and 0.0318 for weeks 1, 2, 3 and 4, respectively.
Compared to placebo, participants supplemented with a composition derived from enriched for one or more phenylpropanoid acids and benzoxazinoids at 500 mg/day showed statistically significant increase in the overall wellbeing on weeks 1 and 3 with P=0.0240, and 0.0028, respectively.
The improvement in the overall wellbeing among groups was statistically significant (p=0.0003).

REM Sleep
Supplementation of a composition derived from enriched for one or more phenylpropanoid acids and benzoxazinoids showed no effect on REM stage of sleep.

TABLE 33
Supplementary Statistical Analysis of the clinical study of
UP165 - a composition derived from enriched for one or more
phenylpropanoid acids and benzoxazinoids. UP165 250 mg/day
- Group A and UP165 500 mg/day - Group B; placebo - Group C).

| | | | | | | Among |
|---|---|---|---|---|---|---|
| | | Week 1 | Week 2 | Week 3 | Week 4 | Groups |
| Parameters | Groups | (p-value) | (p-value) | (p-value) | (p-value) | p-value |
| Deep Sleep | A versus B | 0.1620 | 0.6642 | 0.9982 | 0.9846 | |
| | A versus C | 0.1085 | 0.0219 | 0.0147 | 0.0621 | 0.0318 |
| | B versus C | 0.8470 | 0.0570 | 0.0131 | 0.0554 | |
| REM Sleep | A versus B | 0.6484 | 0.6296 | 0.8836 | 0.8711 | |
| | A versus C | 0.9981 | 0.4600 | 0.4719 | 0.9357 | 0.9065 |
| | B versus C | 0.6551 | 0.7677 | 0.5449 | 0.8075 | |
| Total Sleep | A versus B | 0.4703 | 0.4703 | 0.4703 | 0.4703 | |
| | A versus C | 0.2409 | 0.2409 | 0.2409 | 0.2409 | 0.0006 |
| | B versus C | 0.0501 | 0.0501 | 0.0501 | 0.0501 | |
| Cortisol | A versus B | 0.4598 | 0.7809 | 0.3692 | 0.4227 | |
| | A versus C | 0.4733 | 0.1253 | 0.3143 | 0.1291 | 0.0289 |
| | B versus C | 0.1497 | 0.0757 | 0.0604 | 0.0229 | |
| POMS | A versus B | 0.4198 | 0.2833 | 0.6974 | 0.6233 | |
| Overall | A versus C | 0.0020 | 0.0117 | 0.0006 | 0.0318 | 0.0003 |
| Wellbeing | B versus C | 0.0240 | 0.1585 | 0.0028 | 0.1054 | |
| PSQI | A versus B | 0.2034 | 0.2981 | 0.3491 | 0.9348 | |
| | A versus C | 0.7800 | 0.0690 | 0.0325 | 0.0584 | 0.0273 |
| | B versus C | 0.1231 | 0.4522 | 0.0022 | 0.0756 | |

### Example 20: Second Human Clinical Trial of a Composition Derived from Enriched for One or More Phenylpropanoid Acids and Benzoxazinoids with Measurement of EEG and More Biomarkers

Second human clinical trial has been initiated with larger study population and additional biomarkers to substantiate the clinical study depicted in these examples of inventions. The clinical study protocol has been provided with its entirety from the CRO.

CLINICAL DESIGN: A randomized, triple-blind, placebo controlled, parallel clinical trial to investigate the safety and efficacy of Investigational Product on sleep quality in a healthy population with difficulty falling asleep or staying asleep.

### POPULATION: Healthy Adult Males and Females

SAMPLE SIZE: A sample size has been performed. 80 enrolled participants (40 participants per group)

### CLINICAL TRIAL DESIGN OUTCOMES

Pittsburgh Sleep Quality Index (PSQI)
Profile of Mood States (POMS)
Perceived Stress Scale (PSS)
EEG
Actigraph readings: Sleep Duration, Sleep Onset, Wake Time, Circadian Consistency (0-100), Sleep Latency, Sleep Efficiency, Sleep Stages (awake, light, deep), Awakenings/Disruptions, Movement (w/intensity classification), Snoring (via cellular device microphone, w/intensity classification), Biometric trends: HR, HRV, SpO2, respiratory rate, arterial elasticity, Sleep Score (0-100), Recovery Score (0-100)
Blood markers: Serotonin, melatonin, GABA
Salivary markers: Cortisol
COVID-19 Impact on Quality of Life (QoL) Questionnaire

### SAFETY

Will be as assessed by: Clinical chemistry, hematology, vital sign as well as Adverse Events

### INCLUSION CRITERIA

Males and females between 18 and 65 years of age
Females of childbearing potential must agree to use a medically approved method of birth
control and have a negative urine pregnancy test result
Unable to fall asleep or unable to stay asleep (2 or more waking episodes in a 7-day period)
Agrees to maintain current sleep schedule throughout the study
Agrees to stay in the current time zone for the duration of the study
Agrees to refrain from over-the-counter (OTC) products to help with sleep.
Willingness to complete questionnaires, records and diaries associated with the study and to complete all clinic visits
Provided voluntary, written, informed consent to participate in the study

### EXCLUSION CRITERIA

Women who are pregnant, breast feeding, or planning to become pregnant during the trial
Alcohol or drug abuse in past year
Previous diagnosis of a sleep disorder
Current employment that calls for shift work
Currently experiencing nightmares and/or sleepwalking
Subject has a known allergy to the test material's active or inactive ingredients
Subjects with unstable medical conditions
Clinically significant abnormal laboratory results at screening
Participation in a clinical research trial within 30 days prior to randomization
Individuals who are cognitively impaired and/or who are unable to give informed consent
Any other condition which in the Investigator's opinion may adversely affect the subject's
ability to complete the study or its measures or which may pose significant risk to the subject

### TREATMENTS

Investigational Product—500 mg/day of a composition derived from enriched for one or more phenylpropanoid acids and benzoxazinoids Placebo

VISIT 1 (Screening):

Eligibility will be assessed and determined based on the inclusion and exclusion criteria. A urine pregnancy test will be performed (if applicable). Medical history and concomitant therapies will be reviewed; heart rate, and blood pressure will be measured. Peripheral blood will be collected to determine CBC, electrolytes (Na, K, CI), HbA1c, glucose, eGFR, creatinine, AST, ALT, ALP, and bilirubin. Subjects will begin a 14-day run in period and complete Sleep diary every morning prior to baseline visit.

VISIT 2 (Baseline—Day 0):

Eligible participants will return to the clinic. Sleep diary will be collected and reviewed. Heart rate, and blood pressure will be measured; concomitant therapies will be reviewed. Subjects will be randomized into a treatment group. Pittsburgh Sleep Quality Index (PSQI), Perceived Stress Scale (PSS), the COVID-19 Impact on Quality of Life (QoL) Questionnaire and Profile of Moods (POMS) will be completed. Blood samples were collected to analyze Serotonin, melatonin, GABA. Saliva samples will be collected to measure cortisol levels. Participants will be provided with an Actigraphy device to wear on their wrist to monitor their sleep patterns at night and will be instructed to wear to bed each night. Participants will also be provided with an EEG device and will be trained for at-home use. Investigational product and subject treatment diary will be dispensed, and subjects will be instructed on use. The subject treatment diary will be used to record daily product use, changes in concomitant therapies and any adverse events and symptoms throughout the study.

VISIT 3 (Day 14):

Heart rate, and blood pressure will be measured. Investigational product and subject treatment diary will be returned, and compliance will be calculated. Concomitant therapies and adverse events will be reviewed. Sleep data will be collected from the Actigraphy device and EEG device. PSQI, PSS, the COVID-19 Impact on QoL Questionnaire and POMS will be completed. Blood samples were collected to analyse Serotonin, melatonin, GABA. Saliva samples will be collected to measure cortisol levels. The EEG device will be re-dispensed. Investigational product and subject treatment diary will also be re-dispensed.

VISIT 4 (Day 28—End of Study):

Heart rate and blood pressure will be measured. Investigational product and subject treatment diary will be returned, and compliance will be calculated. Concomitant therapies and adverse events will be reviewed. Sleep data will be collected from the Actigraphy device and EEG device. PSQI, PSS, the COVID-19 Impact on QoL Questionnaire and POMS will be completed. Blood samples will be collected to analyse Serotonin, melatonin, GABA. Saliva samples will be collected to measure cortisol levels. Blood samples will also be collected to determine CBC, electrolytes (Na, K, CI), glucose, eGFR, creatinine, AST, ALT, ALP, and bilirubin

### REFERENCES

1. Balbo M, Leproult R, Van Cauter E. Impact of sleep and its disturbances on hypothalamo-pituitary-adrenal axis activity. Int J Endocrinol. 2010; 2010:759234. doi: 10.1155/2010/759234.
2. Bassett S M, Lupis S B, Gianferante D, Rohleder N, Wolf J M. Sleep quality but not sleep quantity effects on cortisol responses to acute psychosocial stress. Stress. 2015; 18(6):638-44. doi: 10.3109/10253890.2015.1087503
3. Beresford I J, Browning C, Starkey S J, Brown J, Foord S M, Coughlan J, North P C, Dubocovich M L, Hagan R M. GR196429: a nonindolic agonist at high-affinity melatonin receptors. J Pharmacol Exp Ther. 1998 June; 285(3):1239-45. PMID: 9618428.
4. Buysse D J, Reynolds C F 3rd, Monk T H, Berman S R, Kupfer D J. The Pittsburgh Sleep Quality Index: a new instrument for psychiatric practice and research. Psychiatry Res. 1989 May; 28(2):193-213.
5. Carskadon M A, Dement W C 2000 Normal human sleep: an overview. In: Kryger M H, RT, Dement W C, eds. Principles and practice of sleep medicine. Philadelphia: Saunders; 15-25.
6. Deboer T. Sleep homeostasis and the circadian clock: Do the circadian pacemaker and the sleep homeostat influence each other's functioning? Neurobiol Sleep Circadian Rhythms. 2018 Mar. 1; 5:68-77.
7. Durmer J S, Dinges D F. Neurocognitive consequences of sleep deprivation. Semin Neurol. 2005 March; 25(1):117-29. doi: 10.1055/s-2005-867080. PMID: 15798944.
8. Gobbi G, Comai S. Differential Function of Melatonin M T1 and MT2 Receptors in REM and NREM Sleep. Front Endocrinol (Lausanne). 2019 Mar. 1; 10:87.
9. Holsboer F, von Bardeleben U, Steiger A 1988 Effects of intravenous corticotropin-releasing hormone upon sleep-related growth hormone surge and sleep EEG in man. Neuroendocrinology 48:32-38
10. Leproult R, Copinschi G, Buxton O, Van Cauter E. Sleep loss results in an elevation of cortisol levels the next evening. Sleep. 1997 October; 20(10):865-70. PMID: 941594
11. Li J, Vitiello M V, Gooneratne N S. Sleep in Normal Aging. Sleep Med Clin. 2018 March; 13(1):1-11. doi: 10.1016/j.jsmc.2017.09.001. Epub 2017 Nov. 21. PMID: 29412976; PMCID: PMC5841578.
12. Morgan E, Schumm L P, McClintock M, Waite L, Lauderdale D S. Sleep Characteristics and Daytime Cortisol Levels in Older Adults. Sleep. 2017 May 1; 40(5):zsx043.
13. Paul P, Lahaye C, Delagrange P, Nicolas J P, Canet E, Boutin J A. Characterization of 2-[125I]iodomelatonin binding sites in Syrian hamster peripheral organs. J Pharmacol Exp Ther. 1999 July; 290(1):334-40. PMID: 10381796.
14. Rodenbeck A, Cohrs S, Jordan W, Huether G, Rüther E, Hajak G. The sleep-improving effects of doxepin are paralleled by a normalized plasma cortisol secretion in primary insomnia. A placebo-controlled, double-blind, randomized, cross-over study followed by an open treatment over 3 weeks. Psychopharmacology (Berl). 2003 December; 170(4):423-8. doi: 10.1007/s00213-003-1565-0.
15. Rodenbeck A, Huether G, Rüther E, Hajak G. Interactions between evening and nocturnal cortisol secretion and sleep parameters in patients with severe chronic primary insomnia. Neurosci Lett. 2002 May 17; 324(2):159-63.
16. Rosenfeld M J., Forsberg S R. Compounds for use in weight loss and appetite suppression in humans. U.S. Pat. No. 7,507,731. Mar. 24, 2009.
17. Rosenfeld M J., Forsberg S R. Compounds for use in weight loss and appetite suppression in humans. U.S. Pat. No. 7,521,467. Apr. 21, 2009.
18. Rosenfeld M J., Forsberg S R. Compounds for use in weight loss and appetite suppression in humans. U.S. Pat. No. 7,521,468. Apr. 21, 2009.
19. Rosenfeld M J., Forsberg S R. Compounds for use in weight loss and appetite suppression in humans. U.S. Pat. No. 7,524,877. Apr. 28, 2009.
20. Rosenfeld M J., Forsberg S R. Compounds for use in weight loss and appetite suppression in humans. U.S. Pat. No. 7,541,356. Jun. 2, 2009.
21. Rosenfeld M J., Forsberg S R. Compounds for use as antidepressants, aphrodisiacs and adjunctive therapies in humans. U.S. Pat. No. 6,667,308. Dec. 23, 2003.
22. Shelby N J., Godfrey M T., Rosenfeld M J. Methods for inducing anti-anxiety and calming effects in animals and humans. U.S. Pat. No. 7,794,761. Sep. 14, 2010.
23. Stickgold R. Sleep-dependent memory consolidation. Nature. 2005 Oct. 27; 437(7063): 1272-8.
24. Tasali E, Leproult R, Ehrmann D A, Van Cauter E. Slow-wave sleep and the risk of type 2 diabetes in humans. Proc Natl Acad Sci USA. 2008 Jan. 22; 105(3): 1044-9.
25. Van Cauter E, Leproult R, Plat L 2000 Age-related changes in slow wave sleep and REM sleep and relationship with growth hormone and cortisol levels in healthy men. JAMA 284:861-868.
26. van Dalfsen J H, Markus C R. The influence of sleep on human hypothalamic-pituitary-adrenal (HPA) axis reactivity: A systematic review. Sleep Med Rev. 2018 June; 39:187-194.
27. Vgontzas A N, Bixler E O, Lin H M, Prolo P, Mastorakos G, Vela-Bueno A, Kales A, Chrousos G P. Chronic insomnia is associated with nyctohemeral activation of the hypothalamic-pituitary-adrenal axis: clinical implications. J Clin Endocrinol Metab. 2001 August; 86(8):3787-94.
28. Vgontzas A N, Tsigos C, Bixler E O, Stratakis C A, Zachman K, Kales A, Vela-Bueno A, Chrousos G P. Chronic insomnia and activity of the stress system: a preliminary study. J Psychosom Res. 1998 July; 45(1):21-31.
29. Vgontzas A N, Zoumakis M, Bixler E O, Lin H M, Prolo P, Vela-Bueno A, Kales A, Chrousos G P. Impaired nighttime sleep in healthy old versus young adults is associated with elevated plasma interleukin-6 and cortisol levels: physiologic and therapeutic implications. J Clin Endocrinol Metab. 2003 May; 88(5):2087-95.
30. Wheatley D. Medicinal plants for insomnia: a review of their pharmacology, efficacy and tolerability. J Psychopharmacol. 2005 July; 19(4):414-21.
31. Yaneva M, Mosnier-Pudar H, Dugué M A, Grabar S, Fulla Y, Bertagna X. Midnight salivary cortisol for the initial diagnosis of Cushing's syndrome of various causes. J Clin Endocrinol Metab. 2004 July; 89(7):3345-51.

Thus, specific embodiments of compositions and methods for regulating homeostasis of cortisol and improving sleep quality have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification and claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

We claim:

1. A composition comprising an extract, wherein the extract is enriched for one or more phenylpropanoid acids and one or more benzoxazinoids for establishment and regulation of homeostasis of host stress hormone, cortisol, and improvement of sleep quality.

2. The composition of claim 1, wherein the phenylpropanoid acids and the benzoxazinoids in the composition is in a range of 0.05%-99.95% to 99.95%-0.05% by weight of each type of compound with the optimized weight ratio of 40%:60%, respectively.

3. A composition comprising an extract from corn leaf or corn shoots, wherein the extract is enriched for one or more benzoxazinoids comprising both aglycones of benzoxazoles, benzoxazinonoids, benzoxazolinonoids, or glycosides of benzoxazole, benzoxazinonoids, and benzoxazolinonoids

4. The composition of claim 1, wherein one or more phenylpropanoid acids and benzoxazinoids are extracted, enriched and standardized from a plant species selected from the group comprising Zea mays, Oryza species, Oryza sativa, Oryz glaberrima, Oryz australiensis, Oryz brachyantha, Secale cereale, Acanthus arboreus; Acanthus ebracteatus, Acanthus illicifolius, Acanthus mollis, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, Agropyron repens, Blepharis edulis, Balsamocitrus paniculate; Peristrophe roxburghiana; Strobilanthes cusia; Lamium galeobdolon, Lobelia chinensis, Leymus chinensis, Aphelandra spp, Scoparia dulcis, Capparis sikkimensis ssp, or a combination thereof.

5. The composition of claim 1, wherein one or more phenylpropanoid acids and benzoxazinoids are extracted, enriched and standardized from a plant part selected from the group consisting of shoots, germinates from plant seeds, sprouts of geminated grains, immature leaves, mature leaves, whole plants, roots, seeds, flowers, stems, stem barks, root barks, silk, grain, hair roots of germinated grain, stem cells, cell culture tissues or any combination thereof.

6. The composition of claim 1, wherein one or more phenylpropanoid acids and benzoxazinoids are extracted, enriched and standardized from shoots and immature leaves of Zea mays, Zea mays extract, or a combination thereof.

7. The composition of claim 1, wherein phenylpropanoid acid is one or more 7-methoxy-2,7-dihydroxy-2h-1,4-benzoxazin-3(4H)-one; (R)-form 2-O-β-d-glucopyranoside (HMBOA-Glc), 2-hydroxy-2H-1,4-benzoxazin-3(4H)-one; (R)-form, 2,7-dihydroxy-2H-1,4-benzoxazin-3(4H)-one; (R)-form; N-hydroxy-2-hydroxy-2H-1,4-benzoxazin-3(4H)-one; (R)-form, 7-methoxy-2,7-dihydroxy-2H-1,4-benzoxazin-3(4H)-one; (R)-form, N-hydroxy-7-methoxy-2,7-dihydroxy-2H-1,4-benzoxazin-3(4H)-one; (R)-form, cappamensin A, N-methoxy-7-methoxy-2,7-dihydroxy-2H-1,4-benzoxazin-3(4H)-one; (R)-form, monocillinol A, monocillinol B, or any combination thereof.

8. The composition of claim 1, wherein benzoxazinoidis one or more aglycone and glycoside of 6-Methoxy-2-benzoxazolol (MBOA); 2-Benzoxazolol (BOA); 4-Methylbenzoxazole; 2,4-Dimethylbenzoxazole; 2,6-Dimethylbenzoxazole; 2,6-Benzoxazolediol; 2,4-Benzoxazolediol; 4-Acetyl-2(3H)-benzoxazolone; 6-Methoxy-N-methyl-2 (3H)-benzoxazolone; 3-Hydroxy-6-methoxy-2-benzoxazolin-2(3H)-one; 2-Hydroxy-6,7-dimethoxybenzoxazole; 5,6-Dimethoxy-2-benzoxazolinone; 3,6-Dimethoxybenzoxazolin-2(3H)-one; 5-Chloro-6-methoxy-2-benzoxazolinone; Trehalamine or any combination thereof.

9. The composition of claim 1, wherein one or more phenylpropanoid acids and benzoxazinoids in the composition are extracted with any suitable solvent, including supercritical fluid of CO2, water, acidic water, basic water, acetone, methanol, ethanol, propenol, butanol, alcohol mixed with water, mixed organic solvents or a combination thereof.

10. The composition of claim 1, wherein one or more phenylpropanoid acids and benzoxazinoids in the composition are synthesized, metabolized, biodegraded, bioconverted, biotransformed, biosynthesized from small carbon units, by transgenic microbial, by P450 enzymes, by glycotransferase enzyme or a combination of enzymes, or by microbacteria.

11. The composition of claim 1, wherein one or more phenylpropanoid acids and one or more benzoxazinoids in the composition are enriched individually and/or in combination by solvent precipitation, neutralization, solvent partition, ultrafiltration, enzyme digestion, column chromatograph with silica gel, XAD, HP20, LH20, C-18, alumina oxide, polyamide, ion exchange and CG161 resins.

12. The composition of claim 1, wherein the composition further comprises a pharmaceutically or nutraceutically acceptable active, adjuvant, carrier, diluent, or excipient, wherein the pharmaceutical or nutraceutical formulation comprises from about 0.1 weight percent (wt %) to about 99.9 wt % of active compounds in the enriched one or more phenylpropanoid acids and one or more benzoxazinoids composition.

13. The composition of claim 11, wherein the active, adjuvant, excipient or carrier is selected from one or more of Cannabis sativa oil or CBD/THC, turmeric extract or curcumin, terminalia extract, Aloe vera leaf gel powder, Valerian roots, Valeriana officinalis, Ginkgo biloba, Kava kava, Lavender, Passionflower (Passiflora incarnata or maypop), Chamomile flower, Hops, Humulus lupulus, Hibiscus sabdariffa, St. John's Worth, Calif Griffonia simplicifolia, Fermented milks, fish oil, Rhodiola rosea, Lotus seed, Lotus seed germ, Oryza sativa, Zea mays, Ziziphus jujuba, Schisandra chinensis, Magnolia officinalis, Astragalus membranaceus, Ganoderma lucidum, Echinacea purpurea, Echinacea angustifolia, Poria cocos Wolf, Wolfiporia extensa, Withania somnifera, Bupleurum falcatum, Glycyrrhiza spp, Panax quinquefolium, Panax ginseng C. A. Meyer, Korea red ginseng, Eurycoma longifolia (Malaysian ginseng) Lentinula edodes (shiitake), Inonotus obliquus (Chaga mushroom), Melatonin, Magnesium, gamma aminobutyric acid (GABA), vitamin B1, B2, B3, B6, B12, Pyridoxine, Methylcobalamin, Niacinamide, Folic acid, Ascorbic acid, vitamin C, Vitamin D & E, Zinc, omega-3 fatty acid, Glycine, Glutamine, arginine, Tryptophan, L-theanine, 5-Hydroxytryptophan (%-(5-HTP), SAMe, Chlorella, Magnolol, Honokiol, Taurine, Boron, Branched-Chain amino acids (BCAA), phospholipids, phosphatidylserine, phosphatidic acid, theaflavin, rosmarinic acid, catechin, epicatechin, conjugated catechins such as EGCG, ECG, epigallocatechin etc. baicalein, baicalin, Oroxylin, Wogonin, Kaempferol, genistein, quercetin, Butein, Betaine, Luteolin, chrysin, Apigenin, curcumin, resveratrol, glomeratose A, 6-shogaol, gingerol, berberine, piperine.

14. The composition of claim 1, wherein the composition is formulated as a tablet, hard capsule, soft gel capsule, powder, or granule, compressed tablet, pill, gummy, chewing gum, sashay, wafer, bar, or liquid form, tincture, aerial spread, semi solid, semi liquid, solution, emulsion, cream, lotion, ointment, gel base or like form.

15. The composition of claim 1, wherein the route of the administration is selected from the group consisting of oral, topical, suppository, intravenous, intradermic, intragastric, intramuscular, intraperitoneal, or intravenous.

16. The composition of claim 1, wherein one or more phenylpropanoid acids and one or more benzoxazinoids in the composition selectively binds to MT2 over MT1 receptor.

17. The composition of claim 1, wherein one or more phenylpropanoid acids and one or more benzoxazinoids in the composition improves sleep quality by enhancing the deep sleep stage of sleep, increases total sleep time and deep sleep time, improves overall mental well-being measured by the Pittsburgh Sleep Quality Index (PSQI) and Profile of Mood States (POMS), provides positive mood support and enhances emotional well-being; maintains homeostasis of biomarkers—serotonin, melatonin, GABA in formulation in a mammal.

18. The composition of claim 1, wherein one or more phenylpropanoid acids and one or more benzoxazinoids in the composition prevents and treats the most common sleep disorders including insomnia, Hypersomnia, circadian rhythm disorders, shift work sleep disorder, non-24-hour sleep-wake disorder, periodic limb movement disorder, restless legs syndrome (RLS), sleep apnea, narcolepsy, Parasomnias, night terrors, sleepwalking, nightmares, Sleep Eating Disorder. sleep Hallucinations, Sleep Paralysis, Sleep Talking, REM Sleep Behavior Disorder.

19. The composition of claim 1, wherein one or more phenylpropanoid acids and one or more benzoxazinoids in the composition maintains homeostasis of cortisol that leads to improved symptoms of chronically high cortisol including but not limited to anxiety, depression, fatigue, gastrointestinal upset like constipation, bloating, or diarrhea, headache, heart disease, high blood pressure, irritability, problems with memory and concentration, reproductive issues like low libido, erectile dysfunction, or irregular menstruation and ovulation, sleep difficulties, slow recovery from exercise, eating disorders and weight gain.

20. The composition of claim 1, wherein one or more phenylpropanoid acids and one or more benzoxazinoids in the composition is administered at an effective amount of from 0.01 mg/kg to 1000 mg/kg body weight of the mammal.

21. A composition comprising a corn shoot or corn leaf extract, wherein the extract is enriched for one or more phenylpropanoid acids and one or more benzoxazinoids.

22. The composition of claim 1, wherein the extract is from corn leaf or corn shoots.

23. A composition enriched for one or more phenylpropanoid acids and one or more benzoxazinoids for establishment and regulation of homeostasis of host stress hormone, cortisol, and improvement of sleep quality.
